SEQUENCE LISTING (13 sequences)

SEQ ID NO: 1 - DNA, 29 bp, synthetic construct
  source 1..29: mol_type = other DNA; note = P5
aatgatacggcgaccaccgagatctacac

SEQ ID NO: 2 - DNA, 24 bp, synthetic construct
  source 1..24: mol_type = other DNA; note = P7
caagcagaagacggcatacgagat

SEQ ID NO: 3 - DNA, 29 bp, synthetic construct
  source 1..29: mol_type = other DNA; note = P5 prime
gtgtagatctcggtggtcgccgtatcatt

SEQ ID NO: 4 - DNA, 24 bp, synthetic construct
  source 1..24: mol_type = other DNA; note = P7 prime
atctcgtatgccgtcttctgcttg

SEQ ID NO: 5 - protein, 176 aa, synthetic construct
  source 1..176: note = inorganic pyrophosphatase
MSLLNVPAGKDLPEDIYVVIEIPANADPIKYEIDKESGALFVDRFMSTAMFYPCNYGYINHTLSLDGDPVDVLVPTPYPLQPGSVIRCRPVGVLKMTDEAGEDAKLVAVPHSKLSKEYDHIKDVNDLPELLKAQIAHFFEHYKDLEKGKWVKVEGWENAEAAKAEIVASFERAKNK

SEQ ID NO: 6 - protein, 175 aa, synthetic construct
  source 1..175: note = pyrophosphatase
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRACIARYKG

SEQ ID NO: 7 - protein, 176 aa, synthetic construct
  source 1..176: note = pyrophosphatase
MNPFHDLEPGPEVPEVVYALIEIPKGSRNKYELDKKSGLIKLDRVLYSPFYYPVDYGIIPQTWYDDDDPFDIMVIMREPTYPGVLIEARPIGLFKMIDSGDKDYKVLAVPVEDPYFNDWKDISDVPKAFLDEIAHFFQRYKELQGKEIIVEGWENAEKAKQEILRAIELYKEKFKK

SEQ ID NO: 8 - protein, 178 aa, synthetic construct
  source 1..178: note = pyrophosphatase
MNPFHDLEPGPDVPEVVYAIIEIPKGSRNKYELDKKTGLLKLDRVLYSPFFYPVDYGIIPRTWYEDDDPFDIMVIMREPVYPLTIIEARPIGLFKMIDSGDKDYKVLAVPVEDPYFKDWKDIDDVPKAFLDEIAHFFKRYKELQGKEIIVEGWEGAEAAKREILRAIEMYKEKFGKKE

SEQ ID NO: 9 - protein, 176 aa, synthetic construct
  source 1..176: note = pyrophosphatase
MMNLWKDLEPGPNPPDVVYAVIEIPRGSRNKYEYDEERGFFKLDRVLYSPFHYPLDYGFIPRTLYDDGDPLDILVIMQDPTFPGCVIEARPIGLMKMLDDSDQDDKVLAVPTEDPRFKDVKDLDDVPKHLLDEIAHMFSEYKRLEGKETEVLGWEGADAAKEAIVHAIELYEEEHG

SEQ ID NO: 10 - protein, 420 aa, synthetic construct
  source 1..420: note = RB32 UvsX with His tag
MGSSHHHHHHSSGLVPRGSHMSIADLKSRLIKASTSKMTAELTTSKFFNEKDVIRTKIPMLNIAISGAIDGGMQSGLTIFAGPSKHFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDTNCTTFWGPLFKHQPFRDAIKRAYQLGAIDSNEIVEAEVDELINSKVEKFKSPESKSKSAADLETDLEQLSDMEEFNEGGHHHHH

SEQ ID NO: 11 - protein, 393 aa, synthetic construct
  source 1..393: note = RB32 UvsX
MSIADLKSRLIKASTSKMTAELTTSKFFNEKDVIRTKIPMLNIAISGAIDGGMQSGLTIFAGPSKHFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDTNCTTFWGPLFKHQPFRDAIKRAYQLGAIDSNEIVEAEVDELINSKVEKFKSPESKSKSAADLETDLEQLSDMEEFNE

SEQ ID NO: 12 - protein, 398 aa, synthetic construct
  source 1..398: note = Thermophilic UvsX HQ
MSIADLKSRLIKASTSKMTAELTTSKFFNEKDVIRTKIPMLNIAISGAIDGGMQSGLTIFAGPSKSFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDINCTTFWGPLFKHQPFRDAIKRAYQLGAIDSNEIVEAEVDELINSKVEKFKSPESKSKSAADLETDLEQLSDMEEFNEHQHQH

SEQ ID NO: 13 - protein, 400 aa, synthetic construct
  source 1..400: note = Thermophilic UvsX His
MSIADLKSRLIKASTSKMTAELTTSKFFNEKDVIRTKIPMLNIAISGAIDGGMQSGLTIFAGPSKSFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADIVFIIGKRQIKDGSDLQGYQFVLNVEKSRTVKEKSKFFIDVKFDGGIDPYSGLLDMALELGFVVKPKNGWYAREFLDEETGEMIREEKSWRAKDINCTTFWGPLFKHQPFRDAIKRAYQLGAIDSNEIVEAEVDELINSKVEKFKSPESKSKSAADLETDLEQLSDMEEFNEGGHHHHH

(19) United States
(12) Patent Application Publication
(10) Pub. No.: US 2024/0110221 A1
(43) Pub. Date: Apr. 4, 2024

(54) METHODS OF MODULATING CLUSTERING KINETICS

(71) Applicant: Illumina, Inc., San Diego, CA (US)

(72) Inventor: Justin Robbins, San Diego, CA (US)

(73) Assignee: Illumina, Inc., San Diego, CA (US)

(21) Appl. No.: 18/476,052

(22) Filed: Sep. 27, 2023

Related U.S. Application Data
(60) Provisional application No. 63/411,973, filed on Sep. 30, 2022.

(51) Int. Cl. C12Q 1/34; C12Q 1/6844; C12Q 1/6869

(52) U.S. Cl. CPC C12Q 1/34; C12Q 1/6844; C12Q 1/6869

(57) ABSTRACT

This disclosure relates to novel amplification compositions and methods, in particular for use in sequencing.

### CROSS-REFERENCE TO RELATED APPLICATION

This application claims the benefit of U.S. Provisional Patent Application No. 63/411,973, filed Sep. 30, 2022, and entitled “Methods of Modulating Clustering Kinetics,” the disclosure of which is hereby incorporated by reference in its entirety.

### FIELD

This disclosure relates to novel clustering compositions and methods, in particular for use in sequencing.

### SEQUENCE LISTING

The instant application contains a Sequence Listing which has been submitted electronically in xml format and is hereby incorporated by reference in its entirety. Said xml copy was created on Sep. 22, 2023, is named 85491_08600_US.xml, and is 16.4 kilobytes in size.

### BACKGROUND

The detection of analytes such as nucleic acid sequences that are present in a biological sample has been used as a method for identifying and classifying microorganisms, diagnosing infectious diseases, detecting and characterising genetic abnormalities, identifying genetic changes associated with cancer, studying genetic susceptibility to disease, measuring response to various types of treatment and whole exome sequencing to name a few. A common technique for detecting nucleic acid sequences in a biological sample is nucleic acid amplification and sequencing.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing multiple extension reactions using a DNA polymerase to successively incorporate labelled nucleotides to a template strand. In such a “sequencing by synthesis” reaction a new nucleotide strand base-paired to the template strand is built up in the 5′ to 3′ direction by successive incorporation of individual nucleotides complementary to the template strand.

Pyrophosphatases have been described previously for use in sequencing reactions. For example, U.S. Pat. No. 5,744,312 A describes a sequencing composition that comprises a DNA polymerase containing a phenylalanine to tyrosine mutation in combination with a pyrophosphatase. US 2012004115 A1 describes sequencing results using bead-immobilised T. litoralis and Aae PPiase.

However, the development of new clustering compositions can be more challenging, as other factors need to be taken into consideration (e.g. maintaining monoclonality and a high density/intensity of individual clusters).

There remains a need to develop new clustering compositions and methods that can be used to improve clustering and consequently increase throughput and accuracy of sequencing runs. The present disclosure addresses this need.

### SUMMARY

In one aspect of the disclosure, there is provided a clustering composition comprising an inorganic pyrophosphatase (also referred to herein as PPiase).

Preferably, the composition comprises inorganic pyrophosphatase at a concentration of about 0.01 μM to about 1000 μM, about 0.1 μM to about 100 μM, about 0.5 μM to about 50 μM, about 1 μM to about 20 μM, or about 2 μM to about 10 μM.

The composition may further comprise at least one selected from the group consisting of: a recombinase, a single-stranded nucleotide binding protein, a polymerase, nucleotide triphosphates (NTPs), an ATP-generating substrate and an ATP-generating enzyme.

The composition may further comprise at least one selected from the group comprising a recombinase, NTPs and a single stranded nucleotide binding (SSB) protein.

The polymerase may be DNA Polymerase I and the recombinase may be Recombinase A.

The composition may not comprise PEG.

The composition may comprise a buffer, wherein preferably, the composition is buffered to a pH of about 6.0 to about 9.0, preferably about 6.5 to about 8.8, more preferably about 7.5 to about 8.7, even more preferably about 8.3 to about 8.6.

The composition may be a resynthesis composition.

In another aspect, there is provided a thermophilic clustering composition, wherein the composition comprises a thermophilic inorganic pyrophosphatase.

In another aspect, there is provided a mesophilic clustering composition wherein the composition comprises a mesophilic inorganic pyrophosphatase.

In another aspect, there is provided a kit comprising the clustering composition, the thermophilic clustering composition or the mesophilic clustering composition.

The kit may further comprise a metal cofactor composition, preferably wherein the metal cofactor composition comprises magnesium ions.

The clustering composition, the thermophilic clustering composition, the mesophilic clustering composition, or the kit may not comprise primers having a length of between 18 to 22 base pairs.

In another aspect, there is provided the clustering composition, the thermophilic clustering composition or the mesophilic clustering composition to amplify a nucleic acid sequence and/or sequence a nucleic acid sequence.

In another aspect, there is provided a method of amplifying a target nucleic acid template, the method comprising reducing or removing inorganic pyrophosphate during clustering.

In another aspect, there is provided a method of increasing the clustering kinetics of a nucleic acid amplification reaction, the method comprising removing or reducing the levels of inorganic pyrophosphatase.

The method may comprise adding the clustering composition, the thermophilic clustering composition or the mesophilic clustering composition.

The nucleic acid clustering may be performed at a temperature of about 50° C. to about 75° C., preferably about 75° C.

The method may comprise adding the clustering composition only once.

In another aspect, there is provided a method of sequencing a nucleic acid sequence, wherein the method comprises amplifying a nucleic acid template using a method as recited herein; and sequencing the amplified nucleic acid template.

The step of sequencing the amplified nucleic acid template may comprise conducting a first sequencing read and a second sequencing read.

The step of sequencing the amplified nucleic acid template may be conducted using a sequencing-by-synthesis technique or a sequencing-by-ligation technique.

The method may be conducted at temperatures of about 50° C. to about 75° C., preferably about 75° C.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1A shows a typical clustering mixture, such as exclusion amplification (ExAmp). Clustering mixtures typically use four key enzymes to cluster library specific DNA on a solid support, such as flow-cell; a recombinase, a DNA polymerase, a single-stranded DNA binding protein (SSB) and a creatine kinase. FIG. 1B shows the primer extension step. The primer extension step within the RPA (recombinase-polymerase amplification) reaction generates PPi from the DNA polymerase. FIG. 1C shows a reaction scheme for the enzymatic hydrolysis of inorganic pyrophosphate into orthophosphate by inorganic pyrophosphatase. E. coli was used for illustrative purposes in the experiments described herein.

FIG. 2A.) shows five independent clustered HiSeqX v2.5 flowcells post first base incorporation followed by fluorescent imaging by a Typhoon Scanner were sequenced on a HiSeqX instrument to study the impact of PPiase within the clustering formulation under a timecourse. Matched controls for two pushes (Clt-2×30) with the test condition two pushes (PPiase-2×30) incubated for thirty minutes are noted. Matched controls for two pushes (Clt-2×20) with the test condition two pushes (PPiase-2×20) incubated for twenty minutes are noted. FIG. 2B.) Sequence Analysis Viewer (SAV) was utilized to extract the C1 intensity from each independent lane of the sequenced flowcells. The C1 intensity was analyzed with Paired t test and determined with a 95% confidence interval that a statistical difference (****) was observed for the two pushes incubated for twenty minute each in the absence (black circles) and the presence of PPiase (red squares). C1 intensity is measured in relative fluorescence units (RFU). An estimation plot for each lane is show in the right graph pairing each test condition (red circle; presence of PPiase) and the control condition (black circle; absence of PPiase). FIG. 2C.) Sequence Analysis Viewer (SAV) was utilized to extract the C1 intensity from each independent lane of the sequenced flowcells. The C1 intensity was analyzed with Paired t test and determined with a 95% confidence interval that a statistical difference (*) was observed for the two pushes incubated for thirty minutes each in the absence (black circles) and the presence of PPiase (red diamonds). C1 intensity is measured in relative fluorescence units (RFU). An estimation plot for each lane is show in the right graph pairing each test condition (red circle; presence of PPiase) and the control condition (black circle; absence of PPiase). FIG. 2D.) A table showing the average C1 values (reported in RFU) aggregated for all five flowcells. The presence of PPiase at concentration of 1.2 U per 100 μl clustering reagent demonstrated a 12.1% increase in C1 intensity for the two push thirty min (2×30) incubation relative to the absence (Control). The presence of PPiase at concentration of 1.2 U per 100 μl clustering reagent demonstrated a 12.9% increase in C1 intensity for the two push twenty min (2×20) incubation relative to the absence of PPiase (Control). Comparing the two push twenty min incubation in the presence of PPiase at a concentration 1.2 U per 100 μl clustering reagent to two push thirty-minute incubation (2×30) a 1.1% difference in C1 intensity was observed. The two push thirty-minute incubation (2×30) is the standard commercially available clustering recipe. The inclusion of PPiase within the clustering formulation and incubating for two pushes for twenty min attains the level of C1 intensity indicating the kinetics of the clustering has been enhanced in the presence of the PPiase. Under this condition clustering with the same level of intensity as the standard commercially can be achieved in a total of 40 min compared to 60 min for the standard commercially available recipe indicating a 33 percent savings in clustering time. FIG. 2E.) Precision Insertion and Deletion (INDEL) secondary analysis for the two-push thirty-minute incubation (control black bar; 2×30min ctl) compared to the presence of PPiase (red bar; 2×30 min 1.2 U) and two-push twenty-minute incubation (control grey bar; 2×20 min ctl) compared to the presence of PPiase (red bar dashed; 2×20 min 1.2 U). Precision is defined the ability to correctly identify the absence of variants or the absence of false positive (F/P). False positive is defined as a test result that indicates that a person has a specific disease or condition when the person does not have the disease or condition. FIG. 2F.) Recall Insertion and Deletion (INDEL) secondary analysis for the two-push thirty-minute incubation (control black bar; 2×30 min ctl) compared to the presence of PPiase (red bar; 2×30 min 1.2 U) and two-push twenty-minute incubation (control grey bar; 2×20min ctl) compared to the presence of PPiase (red bar dashed; 2×20 min 1.2 U). Recall is defined as the ability to detect variants that are known to be present or the absence of false negative (F/N). False negative is defined as a result that indicates a person does not have a specific disease or condition when the person actually does have the disease or condition. FIG. 2G.) Precision Single Nucleotide Polymorphism (SNP) secondary analysis for the two-push thirty-minute incubation (control black bar; 2×30 min ctl) compared to the presence of PPiase (red bar; 2×30 min 1.2 U) and two-push twenty-minute incubation (control grey bar; 2×20 min ctl) compared to the presence of PPiase (red bar dashed; 2×20 min 1.2 U). Precision is defined the ability to correctly identify the absence of variants or the absence of false positive (F/P). False positive is defined as a test result that indicates that a person has a specific disease or condition when the person does not have the disease or condition. FIG. 2H.) Recall Single Nucleotide Polymorphism (SNP) secondary analysis for the two-push thirty-minute incubation (control black bar; 2×30 min ctl) compared to the presence of PPiase (red bar; 2×30 min 1.2 U) and two-push twenty-minute incubation (control grey bar; 2×20 min ctl) compared to the presence of PPiase (red bar dashed; 2×20 min 1.2 U). Recall is defined as the ability to detect variants that are known to be present or the absence of false negative (F/N). False negative is defined as a result that indicates a person does not have a specific disease or condition when the person actually does have the disease or condition.

FIG. 3A.) Sequence Analysis Viewer (SAV) was utilized to extract the Read 1 (R1) and Read 2 (R2) intensities from the NextSeq 2000 runs. The black bar is R1 or R2 intensity of the control clustering formulation with a standard commercial recipe. The pink bar is R1 or R2 intensity the clustering formulation supplemented with 0.3 U PPiase per 100 μl clustering formulation with the standard modified recipe to pull from the unique well with the cartridge. The red bar is R1 or R2 intensity the clustering formulation supplemented with 1.2 U of PPiase per 100 μl clustering formulation with the standard modified recipe to pull from the unique well with the cartridge. For both read 1 and read 2 the presence of the PPiase increased the intensity. Additionally, the increase in intensity was observed in a concentration dependent manner meaning that in increase in the amount of enzyme utilized increase the intensity signal for both read 1 and read 2. The unit of measure of intensity is relative fluorescence units (RFU). FIG. 3B.) The Quality Score represented in the % Q30 values extracted from SAV. The black bar is the standard clustering formulation; the pink bar is the clustering formulation supplemented with 0.3 U PPiase per 100 μl clustering formulation; the red bar is the clustering formulation 1.2 U PPiase per 100 μl clustering formulation. The % Q30>scores increased in the presence of the PPiase in a concentration dependent manner relative to the control. FIG. 3C.) Instrument yield measured in G output was extracted from SAV. The black bar is the standard clustering formulation; the pink bar is the clustering formulation supplemented with 0.3 U PPiase per 100 μl clustering formulation; the red bar is the clustering formulation 1.2 U PPiase per 100 μl clustering formulation. The yield of NextSeq 2000 increased in the presence of the PPiase in a concentration dependent manner relative to the control. FIG. 3D.) Percent passing filter clusters (% PF) was extracted from SAV. The black bar is the standard clustering formulation; the pink bar is the clustering formulation supplemented with 0.3 U PPiase per 100 μl clustering formulation; the red bar is the clustering formulation 1.2 U PPiase per 100 μl clustering formulation. The % PF of NextSeq 2000 increased in the presence of the PPiase in a concentration dependent manner relative to the control. FIG. 3E.) Recall Single Nucleotide Polymorphism (SNP) secondary analysis for the NextSeq 2000 runs. Recall is defined as the ability to detect variants that are known to be present or the absence of false negative (F/N). False negative is defined as a result that indicates a person does not have a specific disease or condition when the person actually does have the disease or condition. The black bar is the standard clustering formulation; the pink bar is the clustering formulation supplemented with 0.3 U PPiase per 100 μl clustering formulation; the red bar is the clustering formulation 1.2 U PPiase per 100 μl clustering formulation. SNP Recall is unchanged relative to the control under the conditions tested. FIG. 3F.) Precision Single Nucleotide Polymorphism (SNP) secondary analysis for the NextSeq 200 runs. Precision is defined the ability to correctly identify the absence of variants or the absence of false positive (F/P). False positive is defined as a test result that indicates that a person has a specific disease or condition when the person does not have the disease or condition. The black bar is the standard clustering formulation; the pink bar is the clustering formulation supplemented with 0.3 U PPiase per 100 μl clustering formulation; the red bar is the clustering formulation 1.2 U PPiase per 100 μl clustering formulation. SNP Precision is unchanged relative to the control under the conditions tested. FIG. 3G.) Recall Insertion and Deletion (INDEL) secondary analysis for the NextSeq 2000 runs. Recall is defined as the ability to detect variants that are known to be present or the absence of false negative (F/N). False negative is defined as a result that indicates a person does not have a specific disease or condition when the person actually does have the disease or condition. The black bar is the standard clustering formulation; the pink bar is the clustering formulation supplemented with 0.3 U PPiase per 100 μl clustering formulation; the red bar is the clustering formulation 1.2 U PPiase per 100 μl clustering formulation. INDEL Recall is unchanged relative to the control under the conditions tested. FIG. 3H.) Precision Insertion and Deletion (INDEL) secondary analysis for the NextSeq 200 runs. Precision is defined the ability to correctly identify the absence of variants or the absence of false positive (F/P). False positive is defined as a test result that indicates that a person has a specific disease or condition when the person does not have the disease or condition. The black bar is the standard clustering formulation; the pink bar is the clustering formulation supplemented with 0.3 U PPiase per 100 μl clustering formulation; the red bar is the clustering formulation 1.2 U PPiase per 100 μl clustering formulation. INDEL Precision is unchanged relative to the control under the conditions tested.

FIG. 4A.) Four independent clustered HiSeqX v2.5 flowcells post first base incorporation followed by fluorescent imaging by a Typhoon Scanner were sequenced on a HiSeqX instrument to study the impact of PPiase within the clustering formulation in a single push 90-minute recipe configuration. HiSeqXv2.5 flowcell with lanes 1-8 annotated as follows: 1.) 30 min×2 control 2.) 1×90 min with buffer blank 3.) 1×90 min; 4.) 1×90 min; 5.) 1×90 min 6.) 1×90 min with 0.3 U PPiase per 100 ul of clustering reagent); 7.) 1×90 min with 0.3 U PPiase per 100 ul of clustering reagent); 8.) 1×90 min with 0.3 U PPiase per 100 ul of clustering reagent). FIG. 4B.) Sequence Analysis Viewer (SAV) was utilized to extract the C1 intensity from each independent lane of the sequenced flowcells. The C1 intensity was analyzed with Paired t test and determined with a 95% confidence interval that a statistical difference (***) was observed for the 1.2 U PPiase per 100 μl concentration 90-minute incubation (red bar) versus the absence of PPiase clustering reagent when incubated for 90 minutes (grey bar). Additionally, there was no significant difference between the buffer blank 1×90, which contained just the storage buffer and PPiase enzyme, to determine the impact of the carry-over of storage buffer into the clustering formulation (grey bar-dashed) when compared to the 1×90 control (gray bar). This also demonstrates that the PPiase enzyme is driving the changes in the clustering solution under the conditions tested. When compared to the 2×30 min standard recipe control the 1.2 U PPiase per 100 μl concentration 90-minute incubation also had no statistical significance when analyzed. This indicates a similar level of intensity can be obtained in the clustering reaction when incubated at 90 min in the presence of the PPiase in a single push system. Therefore, a single push of clustering reagent when supplemented with the PPiase can achieve a similar level of intensity when two pushes of reagent are utilized, which under the conditions tested would result in reagent reduction and impact COGs.

FIG. 5A.) A clustered HiSeqX v2.5 flowcells post first base incorporation followed by fluorescent imaging by a Typhoon Scanner were sequenced on a HiSeqX instrument to study the impact of PPiase within the clustering formulation in a single push 60-minute recipe configuration while varying the concentration of dNTPs. Lane layout as follows: lane 1: control standard ExAmp (2 pushes at 30 minutes each push); lane 2: ExAmp formulated with 0.3 mM dNTPs; lane 3: ExAmp formulated with 0.3 mM dNTPs & 1.2 U PPiase per 100 μl of ExAmp clustering mix; lane 4: ExAmp formulated with 0.6 mM dNTPs; lane 5: ExAmp formulated with 0.6mM dNTPs & 1.2 U PPiase per 100 μl of ExAmp clustering mix; lane 6: ExAmp formulated with 1.2 mM dNTPs; lane 7: ExAmp formulated with 1.2 mM dNTPs & 1.2 U PPiase per 100 μl of ExAmp clustering mix; lane 8: ExAmp formulated with 2.4 mM dNTPs & 1.2 U PPiase per 100 μl of ExAmp clustering mix. FIG. 5B.) Sequence Analysis Viewer (SAV) was utilized to extract the C1 intensity from each independent lane of the sequenced flowcell. The black bar represents the standard control 2 pushes incubated for 30 min each (2×30 Cont). The gray bars indicate the absence of PPiase in the 60 min time course with a single push of cluster reagent under varying dNTP concentrations. There is a downward trend in C1 intensity as the dNTP concentration is increased within the clustering reaction. However, in the presence of PPiase at 1.2 U per 100 μl clustering formulation an increase in the C1 intensity signal is observed with each test concentration of dNTP in the clustering formulation from 0.3 mM to 1.2 mM. The 2.4 mM dNTP concentration was not graphed because a matched pair was not performed on the flowcell. There is a relationship in the clustering formulations where dNTP concentrations trend with AT coverage in the secondary metric analysis. The addition of the PPiase within the clustering formulation provides a way to mitigate high concentration dNTPs phenotype of low C1 intensity.

### DETAILED DESCRIPTION

The following described features apply to all aspects and embodiments of the disclosure.

The present disclosure is directed to amplification methods and compositions, in particular clustering methods and compositions.

The present disclosure can be used in sequencing, for example pairwise sequencing. Methodology applicable to the present disclosure have been described in WO 08/041002, WO 07/052006, WO 98/44151, WO 00/18957, WO 02/06456, WO 07/107710, W005/068656, U.S. Ser. No. 13/661,524 and US 2012/0316086, the contents of which are herein incorporated by reference. Further information can be found in US 20060024681, US 200602926U, WO 06110855, WO 06135342, WO 03074734, W007010252, WO 07091077, WO 00179553 and WO 98/44152, the contents of which are herein incorporated by reference.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts”. Alternatively “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adaptor sequences.

As used herein an “adapter” sequence comprises a short sequence-specific oligonucleotide that is ligated to the 5′ and 3′ ends of each DNA (or RNA) fragment in a sequencing library as part of library preparation. The adaptor sequence may further comprise non-peptide linkers.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

In one embodiment, the template comprises, in the 5′ to 3′ direction, a first primer-binding sequence (e.g. P5, e.g. SEQ ID NO: 1), an index sequence (e.g. i5), a first sequencing binding site (e.g. SB S3), an insert, a second sequencing binding site (e.g. SBS12), a second index sequence (e.g. i7) and a second primer-binding sequence (e.g. P7′ e.g. SEQ ID NO: 4). In another embodiment, the template comprises, in the 3′ to 5′ direction, a first primer-binding site (e.g. P5′, e.g. SEQ ID NO: 3 which is complementary to P5), an index sequence (e.g. i5′, which is complementary to I5), a first sequencing binding site (e.g. SBS3′ which is complementary to SBS3), an insert, a second sequencing binding site (e.g. SBS12′, which is complementary to SBS12), a second index sequence (e.g. i7′, which is complementary to I7) and a second primer-binding sequence (e.g. P7, E.G. SEQ ID NO: 2 which is complementary to P7′). Either template is referred to herein as a “template strand” or “a single stranded template”. Both template strands annealed together is referred to herein as “a double stranded template”.

A sequence comprising at least a primer-binding sequence (preferably a combination of a primer-binding sequence, an index sequence and a sequencing binding site) may be referred to herein as an adaptor sequence, and a single insert is flanked by a 5′ adaptor sequence and a 3′ adaptor sequence. The first primer-binding sequence may also comprise a sequencing primer for the index read (I5). “Primer-binding sequences” may also be referred to as “clustering sequences” in the present disclosure, and such terms may be used interchangeably.

In a further embodiment, the P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although, in embodiments, the disclosure is not limited to sequences of this length. The precise identity of the amplification primers (e.g. lawn primers), and hence the cognate sequences in the adaptors, are generally not material to the disclosure, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05068656, whose contents are incorporated herein by reference in their entirety. The tag can be read at the end of the first read, or equally at the end of the second read, for example using a sequencing primer complementary to the strand marked P7. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S. 60/899,221. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation is used.

Following denaturation, a single-stranded template library can be contacted in free solution onto a solid support comprising surface capture moieties (for example P5 and P7 lawn primers). This solid support is typically a flowcell, although in alternative embodiments, seeding and clustering can be conducted off-flowcell using other types of solid support.

By way of brief example, following attachment of the P5 and P7 primers to the solid support, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) lead to the formation of (monoclonal) clusters or colonies of template molecules bound to the solid support. This is called clustering.

Thus, solid-phase amplification by either the method analogous to that of WO 98/44151 or that of WO 00/18957 (the contents of which are incorporated herein in their entirety by reference) will result in production of a clustered array comprised of colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582. Further information on amplification can be found in WO0206456 and WO07107710, the contents of which are incorporated herein in their entirety by reference. Through such approaches, a cluster of single template molecules is formed.

To facilitate sequencing, it is preferable if one of the strands is removed from the surface to allow efficient hybridisation of a sequencing primer to the remaining immobilised strand. Suitable methods for linearisation are described in more detail in application number WO07010251, the contents of which are incorporated herein by reference in their entirety.

Sequence data can be obtained from both ends of a template duplex by obtaining a sequence read from one strand of the template from a primer in solution, copying the strand using immobilised primers, releasing the first strand and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′-end. If the first strand is removed from the surface, the sequence of the second strand can be read. This gives a sequence read from both ends of the original fragment.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB/2007/001770, the contents of which are incorporated herein by reference in their entirety. Alternatively, a separate reaction may be carried out containing each of the modified nucleotides added individually.

The modified nucleotides may carry a label to facilitate their detection. In a particular embodiment, the label is a fluorescent label. Each nucleotide type may carry a different fluorescent label. However the detectable label need not be a fluorescent label. Any label can be used which allows the detection of the incorporation of the nucleotide into the DNA sequence. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

Alternative methods of sequencing include sequencing by ligation, for example as described in U.S. Pat. No. 6,306,597 or WO6084132, the contents of which are incorporated herein by reference.

In some embodiments, sequencing may involve pairwise sequencing. The typical steps of pairwise sequencing are known and have been described in WO 2008/041002, the contents of which are herein incorporated by reference. However, the key steps will be briefly described.

The disclosure relates to methods for sequencing two regions of a target double-stranded polynucleotide template, referred to herein as the first and second regions for sequence determination. The first and second regions for sequence determination are at both ends of complementary strands of the double-stranded polynucleotide template, which are referred to herein respectively as first and second template strands. Once the sequence of a strand is known, the sequence of its complementary strand is also known, therefore the term two regions can apply equally to both ends of a single stranded template, or both ends of a double stranded template, wherein a first region and its complement are known, and a second region and its complement are known.

A plurality of template polynucleotide duplexes are immobilised on a solid support. The template polynucleotides may be immobilised in the form of an array of amplified single template molecules, or ‘clusters’. Each of the duplexes within a particular cluster comprises the same double-stranded target region to be sequenced. The duplexes are each formed from complementary first and second template strands which are linked to the solid support at or near to their 5′ ends. Typically, the template polynucleotide duplexes will be provided in the form of a clustered array.

An alternate starting point is a plurality of single stranded templates which are attached to the same surface as a plurality of primers that are complementary to the 3′ end of the immobilised template. The primers may be reversibly blocked to prevent extension. The single stranded templates may be sequenced using a hybridised primer at the 3′ end. The sequencing primer may be removed after sequencing, and the immobilised primers deblocked to release an extendable 3′ hydroxyl. These primers may be used to copy the template using bridged strand resynthesis to produce a second immobilised template that is complementary to the first. Removal of the first template from the surface allows the newly single stranded second template to be sequenced, again from the 3′ end. Thus, both ends of the original immobilised template can be sequenced. Such a technique allows paired end reads where the templates are amplified using a single extendable immobilised primer, for example as described in Polony technology (Nucleic Acids Research 27, 24, e34(1999)) or emulsion PCR (Science 309, 5741, 1728-1732 (2005); Nature 437, 376-380 (2005)).

A critical step in nucleic acid sequencing is amplification, and in particular in the generation of the clusters that comprise an array (or clonal cluster) of amplified template molecules on a solid support. The amplification or clustering reaction typically uses four enzymes, which facilitate clustering, for example through an isothermal system, such as recombinase-polymerase amplification or RPA (Figure la). The reagents required to generate a cluster as described below, are called a clustering composition.

As used herein, the term “cluster” may refer to a group of template polynucleotides (e.g. DNA or RNA) bound within a single well of a flowcell. A “cluster” may contain a sufficient number of copies of a single template polynucleotide such that the cluster is able to output a signal (e.g. a light signal) that allows a single sequencing read to be performed on the cluster. A “cluster” may comprise, for example, about 500 to about 2000 copies, preferably about 600 to about 1800 copies, more preferably about 700 to about 1600 copies, even more preferably about 800 to 1400 copies, yet even more preferably about 900 to 1200 copies, most preferably about 1000 copies of a single template polynucleotide. The copies of the single template polynucleotide may comprise at least about 50%, preferably at least about 60%, more preferably at least about 70%, even more preferably at least about 80%, yet even more preferably at least about 90%, most preferably about 95%, 98%, 99% or 100% of all polynucleotides within a single well of the flowcell, Such monoclonal clusters may be referred to herein as clonal clusters.

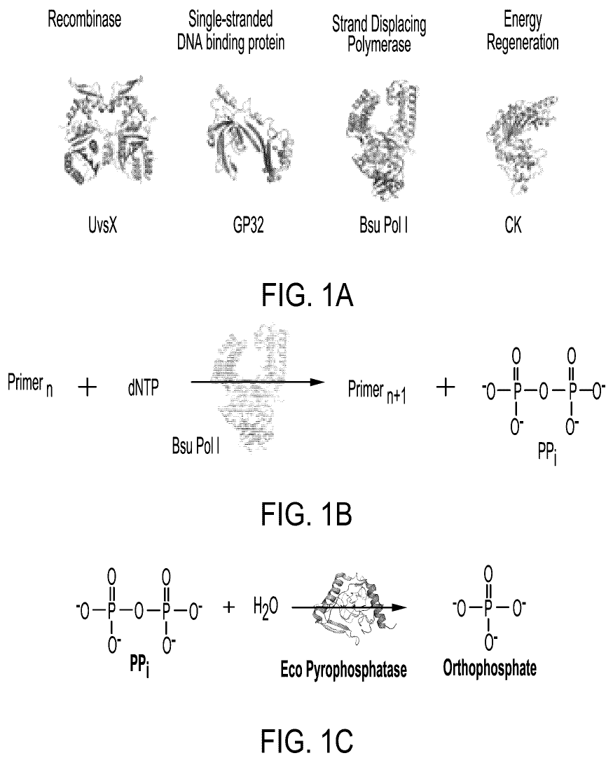

A key step in template amplification is primer extension. Typically this is performed by a polymerase, such as Bacillus subtilus (Bsu) DNA polymerase I (Pol), which generates a by-product called inorganic pyrophosphate (PPi) with each successive NTP (e.g. dNTP) incorporation event (as shown in FIG. 1b). The liberation of PPi is essential to primer extension, however, when it builds up in the aqueous environment of the in vitro reaction it can inhibit the reaction. Accumulation of PPi can also stall the DNA polymerase during strand synthesis, thereby limiting the forward reaction. This is especially problematic when the polymerase encounters secondary structural features within the amplifying DNA strand, such as a G-quadruplex. Stalling of the DNA polymerase can also lead to a phenotype where parts of the library are not clustered and therefore not ultimately sequenced. Finally, the accumulation of PPi can affect the ability of accurately call/detect insertion/deletion events (INDELS) and variants in secondary metrics.

The present disclosure provides a method to remove or reduce the amount of PPi in the DNA clustering reaction. This in turn has been found to improve clustering kinetics and allow the amplification (and subsequent sequencing) of difficult regions of the genome.

Accordingly, in one aspect of the disclosure, there is provided amplification clustering composition comprising means to reduce or remove inhibitory PPi from the system. By “reduce” is meant that the amount or concentration of PPi at any given time point is reduced in a system comprising the composition by at least 10%, 20%, 30%, 40%, 50%, 60%, 70%, 80%, 90%, 95% or 99% compared to a system at the same time point that does not comprise the composition. By “remove” is meant that any PPi generated by the polymerase is removed/converted by the composition such that PPI is not or is barely detectable at any given time point in the system.

Inorganic pyrophosphatases catalyse the hydrolysis of inorganic pyrophosphate to orthophosphate. The reaction scheme is shown in FIG. 1c. Inorganic pyrophosphatase removes the inhibitory PPi, thereby facilitating the primer extension reaction to proceed.

In one embodiment, there is therefore provided a clustering composition comprising an inorganic pyrophosphatase.

As used herein, the term “inorganic pyrophosphatase” (or inorganic diphosphatase) is an enzyme that catalyses the hydrolysis of inorganic pyrophosphate to orthophosphate. The skilled person would understand that the inorganic pyrophosphatase can be derived from any suitable source. In one embodiment, the pyrophosphatase is derived from a yeast or bacteria.

In one embodiment, the pyrophosphatase is derived from a mesophile. Examples of a mesophile include Saccharomyces cerevisiae and E. coli. In one embodiment, the inorganic pyrophosphatase comprises the sequence as shown in SEQ ID NO: 5 or a functional variant or functional fragment thereof.

(SEQ ID NO: 5)
MSLLNVPAGKDLPEDIYVVIEIPANADPIKYEIDKESGAL
FVDRFMSTAMFYPCNYGYINHTLSLDGDPVDVLVPTPYPL
QPGSVIRCRPVGVLKMTDEAGEDAKLVAVPHSKLSKEYDH
IKDVNDLPELLKAQIAHFFEHYKDLEKGKWVKVEGWENAE
AAKAEIVASFERAKNK

In another embodiment, the pyrophosphatase is derived from a thermophile (including a hyperthermophile). Examples of thermophiles or hyperthermophile include microbes from the family Thermococcaceae, Thermaceae or Thermotogaceae; or from the genus Thermus, the genus Meiothermus, the genus Thermococcus, the genus Pyrococcus, the genus Methanopyrus or the genus Thermotoga. In one embodiment, the thermophile may be selected from Thermococcus kodacaraensis, Pyrococcus abyssi, Pyrococcus furiosus, Pyrococcus species GB-D, Pyrococcus woesei, Meiothermus ruber, Thermus aquaticus, Thermus brokianus, Thermus caldophilus, Thermus filiformis, Thermus flavus, Thermococcus fumiculans, Thermococcus gorgonarius, Thermococcus litoralis, Thermotoga maritima, Thermotoga neopolitana and Thermus thermophilus.

In one embodiment, the thermophile is from the genus Thermus. In one embodiment, the thermophile is Thermus thermophilus and the pyrophosphatase may comprise the following sequence or a functional variant or functional fragment thereof:

(SEQ ID NO: 6)
MANLKSLPVGDKAPEVVHMVIEVPRGSGNKYEYDPDLGAI
KLDRVLPGAQFYPGDYGFIPSTLAEDGDPLDGLVLSTYPL
LPGVVVEVRVVGLLLMEDEKGGDAKVIGVVAEDQRLDHIQ
DIGDVPEGVKQEIQHFFETYKALEAKKGKWVKVTGWRDRK
AALEEVRACIARYKG

In another embodiment, the thermophile is from the genus Thermococcus. In one embodiment, the thermophile is Thermococcus litoralis and the pyrophosphatase may comprise the following sequence or a functional variant or functional fragment thereof:

(SEQ ID NO: 7)
MNPFHDLEPGPEVPEVVYALIEIPKGSRNKYELDKKSGLI
KLDRVLYSPFYYPVDYGIIPQTWYDDDDPFDIMVIMREPT
YPGVLIEARPIGLFKMIDSGDKDYKVLAVPVEDPYFNDWK
DISDVPKAFLDEIAHFFQRYKELQGKEIIVEGWENAEKAK
QEILRAIELYKEKFKK

In another embodiment, the thermophile is from the genus Pyrococcus. In one embodiment, the thermophile is Pyrococcus furiosus and the pyrophosphatase may comprise the following sequence or a functional variant or functional fragment thereof:

(SEQ ID NO: 8)
MNPFHDLEPGPDVPEVVYAIIEIPKGSRNKYELDKKTGLL
KLDRVLYSPFFYPVDYGIIPRTWYEDDDPFDIMVIMREPV
YPLTIIEARPIGLFKMIDSGDKDYKVLAVPVEDPYFKDWK
DIDDVPKAFLDEIAHFFKRYKELQGKEIIVEGWEGAEAAK
REILRAIEMYKEKFGKKE

In another embodiment, the thermophile is from the genus Methanopyrus. In one embodiment, the thermophile is Methanopyrus kandleri and the pyrophosphatase may comprise the following sequence or a functional variant or functional fragment thereof:

(SEQ ID NO: 9)
MMNLWKDLEPGPNPPDVVYAVIEIPRGSRNKYEYDEERGF
FKLDRVLYSPFHYPLDYGFIPRTLYDDGDPLDILVIMQDP
TFPGCVIEARPIGLMKMLDDSDQDDKVLAVPTEDPRFKDV
KDLDDVPKHLLDEIAHMFSEYKRLEGKETEVLGWEGADAA
KEAIVHAIELYEEEHG

In one embodiment, the clustering composition comprises inorganic pyrophosphatase at a concentration of about 0.01 μM to about 1000 μM, about 0.1 μM to about 100 μM, about 0.5 μM to about 50 μM, about 1 μM to about 20 μM, or about 2 μM to about 10 μM. Alternatively, the composition comprises between about 0.01 U/μL and about 100 U/μL of the inorganic pyrophosphatase, between about 0.1 U/μL and about 50 U/μL, between about 0.2 U/μL and about 30 U/μL, between about 0.3 U/μL and about 20 U/μL, between about 0.5 U/μL and about 10 U/μL, or between about 1.0 U/μL and about 5.0 U/μL. For example, the composition may comprise around 0.3 U/μL, 0.4 U/μL, 0.5 U/μL, 0.6 U/μL, 0.7 U/μL, 0.8 U/μL, 0.9 U/μL, 1.0 U/μL, 1.1 U/μL, 1.2 U/μL, 1.3 U/μL, 1.4 U/μL, 1.5 U/μL, 1.6 U/μL, 1.7 U/μL, 1.8 U/μL, 1.9 U/μL or around 2.0 U/μL of the inorganic pyrophosphatase. In one embodiment, the composition comprises between about 0.3 U per 100 μl of the clustering composition. In another embodiment, the composition comprises between about 1.2 U per 100 μl of the clustering composition. Alternatively, the inorganic pyrophosphatase is present at a wt % between about 0.01 wt % to about 5.0 wt %, about 0.02 wt % to about 4.5 wt %, about 0.05 wt % to about 4.0 wt %, about 0.08 wt % to about 3.5 wt %, about 0.1 wt % to about 3.0 wt %, about 0.2 wt % to about 2.5 wt %, or about 0.5 wt % to about 2.0 wt % with respect to a total wt % of the composition by dry mass.

As used herein, the term “inorganic pyrophosphate” (or “PPi”) may refer to two phosphate residues connected by a phosphoanhydride bond.

An inorganic pyrophosphate may be present in an acid form, a salt form, or a combination thereof. In cases where the inorganic pyrophosphate is present in a salt form, the inorganic pyrophosphate may comprise a cation (not including H⁺). For example, the cation may be selected from “metal cations” or “non-metal cations”. Metal cations may include alkali metal ions (e.g. lithium, sodium, potassium, rubidium or caesium ions). Non-metal cations may include ammonium salts (e.g. alkylammonium salts) or phosphonium salts (e.g. alkylphosphonium salts).

The inorganic pyrophosphate may be soluble in aqueous medium.

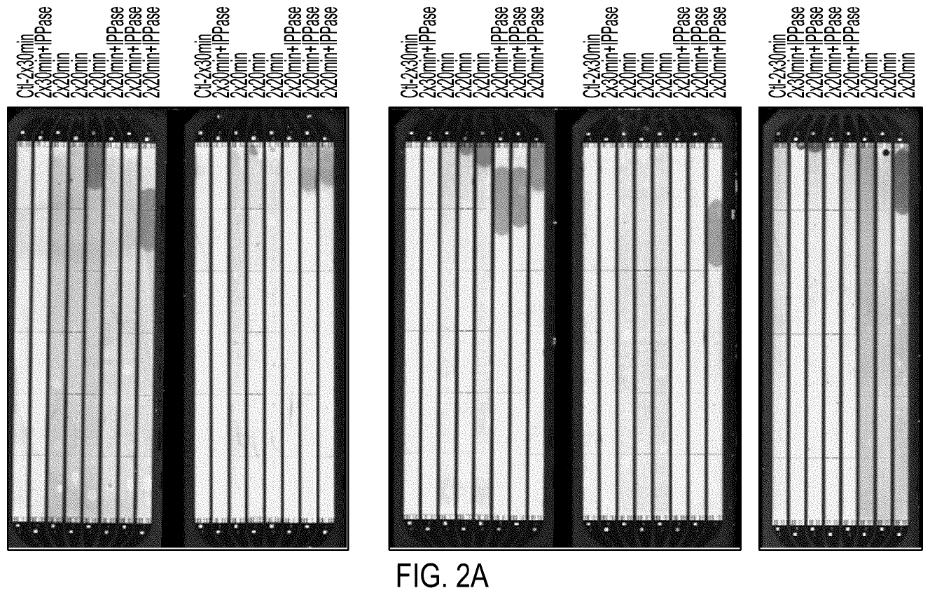

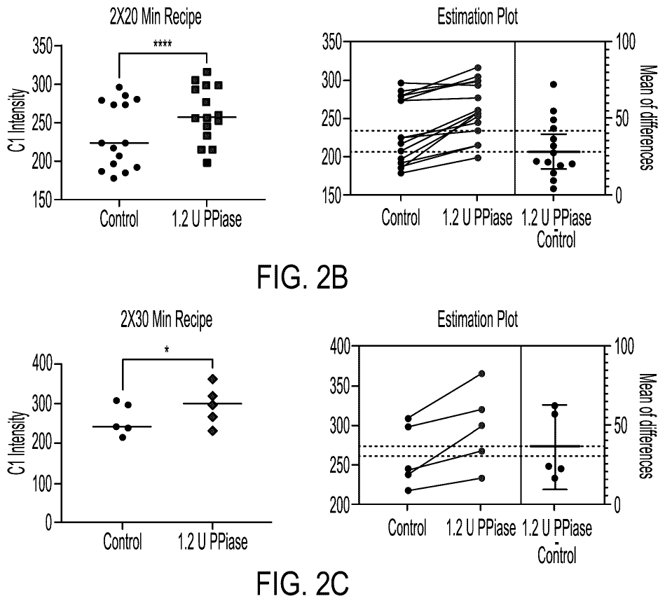

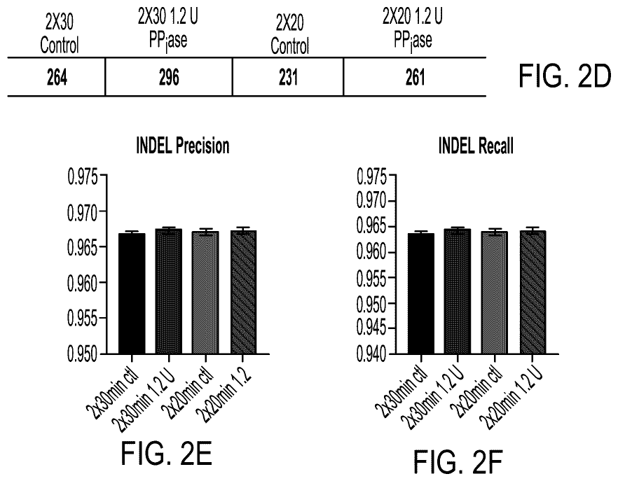

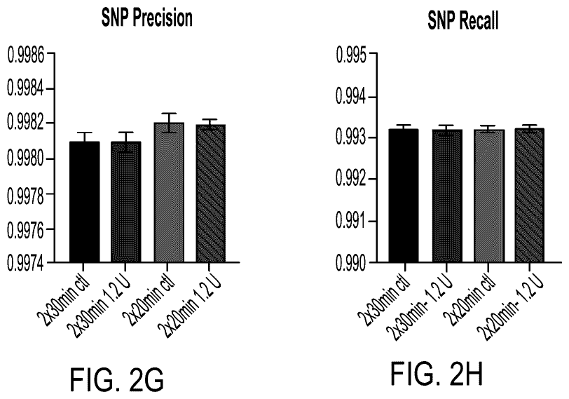

The present inventor found that the removal of inorganic pyrophosphate during clustering, for example by the addition of inorganic pyrophosphatase, has a number of advantages in methods of cluster generation and subsequently sequencing. First, the addition of inorganic pyrophosphatase improves clustering kinetics. In an amplification or clustering reaction it may be necessary to add the amplification composition more than once (the number of times the amplification composition is added to the flowcell may be called a “push”). By “clustering kinetics” is meant the rate at which a clonal cluster of amplified target sequence generates over a defined period of time—e.g. at least 60 minutes total incubation time (with 30 min per push and a minimum of two pushes of ExAmp are utilized) is a typical time to perform clustering. Increasing cluster density is particularly important in NGS sequencing as the density of the clonal cluster has a large impact on sequencing performance (e.g. data quality and total data output). Increasing cluster kinetics also in turn leads to a decrease in clustering time (i.e. the time it takes to generate a (clonal) cluster or amplify a given target sequence). This is shown, for example, in FIG. 2. Here, inorganic pyrophosphatase was added to the composition for different periods of time: 30 minutes and 20 minutes. As can be seen in FIGS. 2B, C and D, the addition of inorganic pyrophosphatase increased the signal intensity at all time points tested compared to lanes without inorganic pyrophosphatase. This increase in intensity was most apparent at 20 minutes, which resulted in equal intensity as the control, which is the clustering reaction under standard conditions (FIG. 2D). These results show that it is possible to achieve the same level of clustering (or clonal amplification of a target sequence) in about 40 minutes (compared to 60 minutes) with the addition of inorganic pyrophosphatase—a saving of ˜33%. This leads to faster end-to-end turnaround times for the user. The standard time for clustering is 30 minutes. These results also show that after 30 minutes the signal intensity was much higher than the signal intensity in the lane without inorganic pyrophosphatase at 30 minutes. This result in turn boosts the sequencing signal to noise ratio over standard methods.

Thus, this data shows that the addition of inorganic pyrophosphatase can be used to improve clustering kinetics, and in turn reduce clustering times (and thus turnaround times) and/or increase the signal intensities (and thus increase the sequence signal:noise ratios).

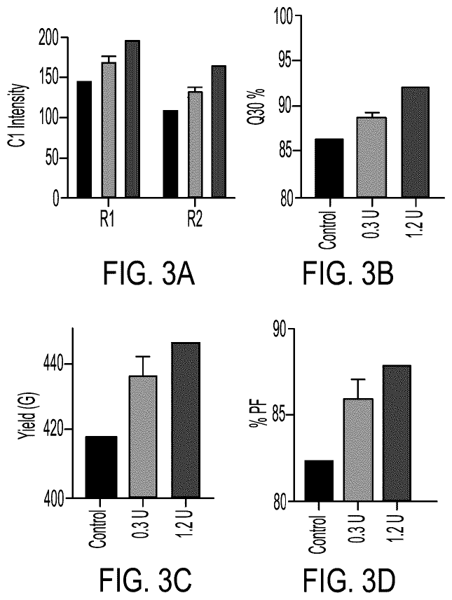

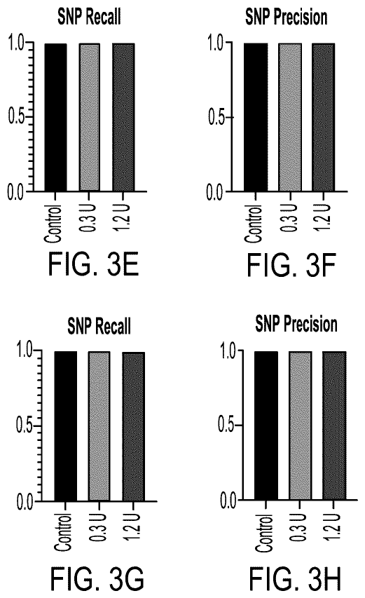

Improving clustering kinetics by the removal or reduction of PPi also leads to improvements in sequencing performance. As shown in FIGS. 3A-3H, addition of inorganic pyrophosphatase at 0.3 U and 1.2 U increasing the intensity, % PF, Q30 and Yield (g). By “% PF” is meant the % of reads that pass the chastity filter (chastity is the ratio is the ratio of the brightest base intensity divided by the sum of the brightest and second brightest base intensities”). By “Q30” is meant The percentage of bases with a quality score of 30 or higher. A quality score is “an estimate of the probability of that base being called wrongly: q=−10×log 10(p)”. By “yield” is meant the number of bases generated in the run.

Second, increasing clustering intensity also allows amplification/clustering to take place in smaller wells, where a decrease in well size requires an increase in signal intensity.

Third, these results were achieved without any detrimental effect on secondary metrics, as for example shown in FIGS. 2E, 2F, 3G and 3H. In these Figures, INDEL and SNP Recall (the ability to detect variants that are known to be present or the absence of a false negative) and Precision (the ability to correctly identify the absence of variants or the absence of a false positive) was measured with and without the addition of inorganic pyrophosphatase. As shown in FIGS. 2E, 2F, 3G and 3H the INDEL recall and precision and SNP recall and precision levels were similar or identical to control (no addition of inorganic pyrophosphatase).

Fourth, as explained above, the accumulation of inorganic pyrophosphate stalls DNA polymerases (e.g. DNA polymerases). This is problematic where the DNA polymerase encounters structured secondary features like a G-quadruplex, leading to parts of the library that are not clustered and therefore not sequenced. Removal of inorganic pyrophosphate reduces the likelihood or prevents stalling of the polymerase, and consequently a decrease in sequence specific errors because the polymerase is able to cluster/structured regions of the genome.

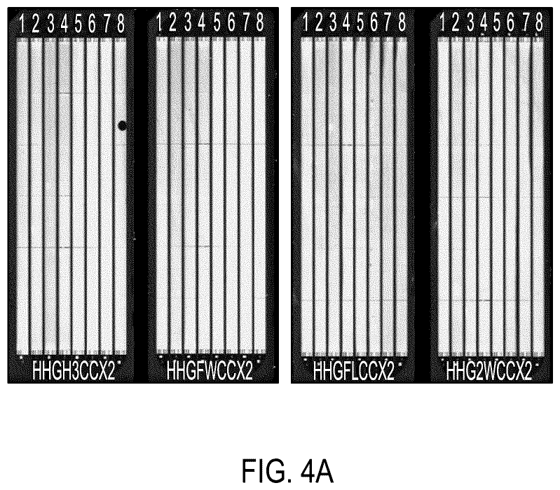

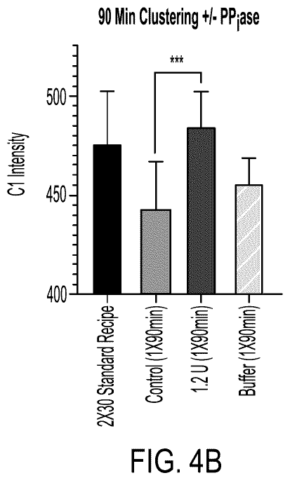

Fifth, in addition to improving clustering kinetics (e.g. clustering times and the signal intensity), the addition of inorganic pyrophosphatase can also significantly reduce the amount of clustering reagents needed by as much as 50%. As mentioned, in an or clustering reaction it may be necessary to add the composition more than once (the number of times the amplification composition is added to the flowcell may be called a “push”). Multiple pushes may be necessary to achieve the required level of sequence signal intensity. The present inventor has found that removal of inorganic pyrophosphate significantly increases the sequence signal intensity with a single push. This is shown in FIG. 4. In FIG. 4, the C1 signal intensity of a 2×30 minute push of the amplification composition was compared to single push of a composition with inorganic pyrophosphatase added. As shown in this Figure, the addition of inorganic pyrophosphatase significantly increased the C1 intensity compared to control (no inorganic pyrophosphatase added), and, of note, increased the C1 intensity compared to the 2×30 minute push. Therefore, as shown in FIG. 4, by increasing the incubation time to 90 minutes, it is possible to obtain intensity values with a single push of the composition comprising inorganic pyrophosphatase better than the standard double-push of the composition for 30 minutes. Accordingly, by reducing PPi levels it is possible to additionally half the amount of composition needed (i.e. half the COGs (cost of goods) without affecting clustering/intensities.

By “amplification composition” is meant a composition that is suitable for the amplification of a target nucleic acid template. By contrast, a “cluster composition” refers to a composition that is suitable for the amplification of a (single) target sequence into a cluster (i.e. the composition is suitable for cluster generation, particularly for the generation of a monoclonal cluster) as described above, not just for any amplification method. In one embodiment, the composition is not additionally suitable for the detection or sequencing of the nucleic acid template. For example, in one embodiment, the composition does not comprise a fluorescent entity, such as probes, nucleotides labelled with a fluorescent entity, and/or primers labelled with a fluorescent entity. Alternatively, the composition does not comprise leuco dyes/reagents labelled with leuco dyes.

In one embodiment, the composition may be a resynthesis composition. By resynthesis is meant the step between the first and second sequencing reads where the template is copied using bridged strand resynthesis to produce a second immobilised template that is complementary to the first. Accordingly, the same composition as described herein may be used in resynthesis.

The composition may further comprise a recombinase. The recombinase may be a thermophilic recombinase.

As used herein, the term “recombinase” may refer to an enzyme which can facilitate invasion of a target nucleic acid by a polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolysable analogs thereof) in a recombinase-facilitated amplification reagent to facilitate amplification. A mixture of recombinase and single-stranded binding (SSB) protein is particularly useful as SSB can further facilitate amplification. Recombinases may include, for example, RecA protein, the T4 uvsX protein, any homologous protein or protein complex from any phyla, or functional variants thereof. Eukaryotic RecA homologues are generally named Rad51 after the first member of this group to be identified. Other non-homologous recombinases may be utilised in place of RecA, for example, RecT or RecO.

In some preferred embodiments, the recombinase may be UvsX. In one embodiment, the UvsX comprises or consists of SEQ ID NO: 5 or 6 or a functional fragment or functional variant thereof.

In other preferred embodiments, the recombinase may be a thermophilic UvsX. In one embodiment, the thermophilic UvsX comprises or consists of SEQ ID NO: 7 or 8 or a functional fragment or functional variant thereof.

The composition may further comprise a single-stranded nucleotide binding protein.

As used herein, the term “single-stranded nucleotide binding protein” may refer to any protein having a function of binding to a single stranded nucleic acid, for example, to prevent premature annealing, to protect the single-stranded nucleic acid from nuclease digestion, to remove secondary structure from the nucleic acid, or to facilitate replication of the nucleic acid. The term is intended to include, but is not necessarily limited to, proteins that are formally identified as Single Stranded Binding proteins by the Nomenclature Committee of the International Union of Biochemistry and Molecular Biology (NC-IUBMB). Exemplary single stranded binding proteins include, but are not limited to E. coli SSB, T4 gp32, T7 gene 2.5 SSB, phage phi 29 SSB, any homologous protein or protein complex from any phyla, or functional variants thereof.

The composition may further comprise a polymerase. Preferably, the polymerase may be a strand-displacing polymerase. In some preferred embodiments, the polymerase may be a DNA polymerase. In other preferred embodiments, the polymerase may be a RNA polymerase. The polymerase may be a thermophilic polymerase.

As used herein, the term “polymerase” may refer to an enzyme that produces a complementary replicate of a nucleic acid molecule using the nucleic acid as a template strand. Typically, DNA polymerases bind to the template strand and then move down the template strand sequentially adding nucleotides to the free hydroxyl group at the 3′ end of a growing strand of nucleic acid. DNA polymerases typically synthesise complementary DNA molecules from DNA templates and RNA polymerases typically synthesise RNA molecules from DNA templates (transcription). Polymerases can use a short RNA or DNA strand, called a primer, to begin strand growth. Some polymerases can displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

The composition may further comprise a nucleotide triphosphate (NTP). Preferably, the nucleotide triphosphate may be a deoxynucleotide triphosphate (dNTP). More preferably, the composition comprises a plurality of NTPs or dNTPs, and preferably a mixture—for example comprising a plurality of dATP, dGTP, dCTP and dTTP for DNA clustering/synthesis or ATP, GTP, CTP and UTP for RNA clustering/synthesis. In one embodiment, the concentration of dNTPs may be between 0.1 and 2 mM, preferably between 0.2 to 1.5 mM, more preferably between 0.3 to 1.2 mM, even more preferably between 0.3 to 0.6 mM; for example, the concentration may be selected from 0.3 mM, 0.6 mM and 1.2 mM.

As used herein, the term “nucleotide triphosphate” may refer to a molecule containing a nitrogenous base (e.g. adenine, thymine, cytosine, guanine, uracil) bound to a 5-carbon sugar (e.g. ribose or deoxyribose), with three phosphate groups bound to the sugar.

As used herein, the term “deoxynucleotide triphosphate” or (dNTPs) may refer to a molecule containing a nitrogenous base (e.g. adenine, thymine, cytosine, guanine, uracil) bound to deoxyribose, with three phosphate groups bound to the deoxyribose.

The composition may further comprise an ATP-generating substrate.

As used herein, the term “ATP-generating substrate” may refer to any substrate that is able to react with ADP to form ATP. Examples of ATP-generating substrates include creatine phosphate (CP).

The composition may further comprise an ATP-generating enzyme.

As used herein, the term “ATP-generating enzyme” may refer to any enzyme that is able to catalyse a reaction of ADP to form ATP. Examples of ATP-generating enzymes include creatine kinase.

The ATP-generating substrate as described herein may be paired with an appropriate ATP-generating enzyme that catalyses the reaction of that ATP-generating substrate with ADP to form ATP. Thus, in some preferred embodiments, the composition may comprise creatine phosphate (CP) and creatine kinase.

In some embodiments, the composition may not comprise creatine kinase and/or creatine phosphate.

The composition may comprise at least one selected from the group consisting of: a recombinase, a single-stranded nucleotide binding protein, a polymerase, nucleotide triphosphates (NTPs), an ATP-generating substrate and an ATP-generating enzyme. Preferably, the composition may comprise at least two selected from the group consisting of: a recombinase, a single-stranded nucleotide binding protein, a polymerase, nucleotide triphosphates (NTPs), an ATP-generating substrate and an ATP-generating enzyme. More preferably, the composition may comprise at least three selected from the group consisting of: a recombinase, a single-stranded nucleotide binding protein, a polymerase, nucleotide triphosphates (NTPs), an ATP-generating substrate and an ATP-generating enzyme. Even more preferably, the composition may comprise at least four selected from the group consisting of: a recombinase, a single-stranded nucleotide binding protein, a polymerase, nucleotide triphosphates (NTPs), an ATP-generating substrate and an ATP-generating enzyme. Yet even more preferably, the composition may comprise at least five selected from the group consisting of: a recombinase, a single-stranded nucleotide binding protein, a polymerase, nucleotide triphosphates (NTPs), an ATP-generating substrate and an ATP-generating enzyme.

Preferably, the composition further comprises at least one selected from the group comprising a recombinase, NTPs and a single stranded nucleotide binding (SSB) protein. More preferably, the composition further comprises at least two selected from the group comprising a recombinase, NTPs and a single stranded nucleotide binding (SSB) protein.

Preferably, the composition may comprise a recombinase, NTPs and a single stranded nucleotide binding (SSB) protein.

Preferably, the composition may comprise a recombinase, a single-stranded nucleotide binding protein, a polymerase, nucleotide triphosphates (NTPs), an ATP-generating substrate and an ATP-generating enzyme.

In some embodiments, the composition may not comprise one or more primers, either an amplification or a sequencing primer. Accordingly, the composition may not comprise primers. That is, the composition may not comprise any nucleic acid sequences that can initiate DNA synthesis (by a polymerase). The primers may be free nucleic acid sequence of between 18 and 22 base pairs, more preferably between 15 to 30 base pairs. The GC content of the free nucleic acid sequence may also be between 50 and 55%, and preferably, may have a GC-lock (a G or C in the last 5 bases of the sequence) at the 3′ end. The melting temperature of the primers may be between 40 and 60° C., more preferably between 50 and 55° C. The primers may also be complementary or substantially complementary (with e.g. at least 80% overall sequence identity) to a target sequence or complement thereof that the composition is intended to cluster. The primers may also comprise one or more restriction sites.

In some embodiment, the composition may also comprise a nucleic acid template. The nucleic acid template may also comprise the adaptor sequences described herein, where preferably the adaptor sequences comprise at least one of P5, P5′, P7 and P7′, the sequences of which are described below.

In another aspect, there is provided a thermophilic clustering composition, wherein the composition comprises a thermophilic inorganic pyrophosphatase. The thermophilic inorganic pyrophosphatase may be derived from a thermophilic organism as described above.

In a further embodiment, where the composition comprises at least one (preferably all of) of a recombinase, a single-stranded DNA binding protein, a strand displacing polymerase and a form of energy regeneration.

As used herein, the term “thermophilic” or “thermostable” may refer to a protein that does not substantially denature at high temperature, for example above 40° C., above 45° C., above 50° C., above 55° C., above 60° C., above 65° C., above 70° C., above 75° C., above 80° C., above 85° C., above 90° C., above 95° C., or above 100° C.

The inorganic pyrophosphatase, may have an optimum working temperature of about 50° C. to about 75° C., about 55° C. to about 70° C., or about 60° C. to about 65° C.

The thermophilic composition may be used in thermophilic clustering. Thermophilic clustering can leverage elevating the clustering reaction to 75° C. to take advantage of enhanced kinetic rates due to the Arrhenius equation. Therefore, increased kinetics has the potential to decrease the clustering times.

In another aspect, there is provided a mesophilic clustering composition, wherein the composition comprises a mesophilic inorganic pyrophosphatase. The mesophilic inorganic pyrophosphatase may be derived from a mesophilic organism, such as Saccharomyces cerevisiae or E. coli as described above.

As used herein, the term “mesophile” may refer to a protein that does not substantially denature at moderate temperatures, for example, between about 20° C. and about 45° C. These proteins may have an optimum activity in the range of about 30° C. to about 40° C.

Accordingly, in an alternative embodiment, the inorganic pyrophosphatase, may have an optimum working temperature of about 30° C. to about 40° C., preferably about 32° C. to about 39° C., more preferably about 34° C. to about 38° C.

As used herein, the term “optimum working temperature” may refer to a temperature at which the catalytic activity of the enzyme reaches a peak maximum value.

As used herein, the term “functional variant” refers to a variant polypeptide sequence or part of the polypeptide sequence which retains the biological function of the full non-variant sequence. For example, a functional variant of inorganic pyrophosphatase is able to catalyse the hydrolysis of inorganic pyrophosphate to orthophosphate.

A functional variant also comprises a variant of the polypeptide of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a polypeptide sequence that does not affect the functional properties of the polypeptide are well known in the art. For example, the amino acid alanine, a hydrophobic amino acid, may be substituted by another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

As used in any aspect described herein, a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant amino acid sequence and preferably retains the catalytic activity of the inorganic pyrophosphatase as described above. The sequence identity of a variant can be determined using any number of sequence alignment programs known in the art. As an example, Emboss Stretcher from the EMBL-EBI may be used: https://www.ebi.ac.uk/Tools/psa/emboss_stretcher/ (using default parameters: pair output format, Matrix=BLOSUM62, Gap open=1, Gap extend=1 for proteins; pair output format, Matrix=DNAfull, Gap open=16, Gap extend=4 for nucleotides).

As used herein, the term “functional fragment” refers to a functionally active series of consecutive amino acids from a longer polypeptide or protein. For example, a functional fragment may retain the catalytic activity of the inorganic pyrophosphatase as described above.

In one embodiment, the composition may not comprise PEG.

In another embodiment, the composition may also or alternatively not comprise luciferase and/or apyrase and/or luciferin.

The amplification composition may comprise a buffer. Preferably, the amplification composition is buffered to a pH of about 6.0 to about 9.0, preferably about 6.5 to about 8.8, more preferably about 7.5 to about 8.7, even more preferably about 8.3 to about 8.6.

The composition may be supplied in a dry form (e.g. a freeze-dried form or a lyophilised form). In such a case, the composition may be rehydrated, for example with water or a buffer solution, prior to use in clustering. In other embodiments, the composition may be supplied as a solution (e.g. as an aqueous solution).

The composition may further comprise excipients. Suitable excipients may include surfactants, such as anionic surfactants, including alkyl sulfates (e.g. ammonium lauryl sulfate, sodium lauryl sulfate, sodium laureth sulfate, sodium myreth sulfate, sodium docusate), alkyl sulfonates (e.g. perfluorooctanesulfonate, perfluorobutanesulfonate), alkyl phosphates (e.g. alkyl-aryl ether phosphates, alkyl ether phosphates) and alkyl carboxylates (e.g. sodium stearate, sodium lauroyl sarcosinate, perfluorononanoate, perfluorooctanoate); cationic surfactants, including quaternary ammonium salts (e.g. cetrimonium bromide, cetylpyridinium chloride, benzalkonium chloride, benzethonium chloride, dimethyldioctadecylammonium chloride, dioctadecyldimethylammonium bromide); non-ionic surfactants, including fatty alcohol ethoxylates, alkylphenol ethoxylates, fatty acid ethoxylates, ethoxylated amines or fatty acid amides, poloxamers, polysorbates, (e.g. polyethylene glycol sorbitan alkyl esters (Tween)). Further excipients may include enzyme stabilisers, such as dithiothreitol (DTT), tris(2-carboxyethyl)phosphine (TCEP) and 2-mercaptoethanol (BME). Still further excipients may include molecular crowding agents such as polyethylene glycol (PEG), dextrans and epichlorohydrin-sucrose polymers (e.g. Ficoll); in some embodiments, PEG may be excluded.

In a further aspect, the present disclosure is directed to a kit comprising a clustering composition, the clustering composition comprising an inorganic pyrophosphatase.

In some embodiments, the composition may not comprise one or more primers, either an amplification or a sequencing primer. Accordingly, the composition may not comprise primers. That is, the composition may not comprise any nucleic acid sequences that can initiate DNA synthesis (by a polymerase). The primers may be free nucleic acid sequence of between 18 and 22 base pairs, more preferably between 15 to 30 base pairs. The GC content of the free nucleic acid sequence may also be between 50 and 55%, and preferably, may have a GC-lock (a G or C in the last 5 bases of the sequence) at the 3′ end. The melting temperature of the primers may be between 40 and 60° C., more preferably between 50 and 55° C. The primers may also be complementary or substantially complementary (with e.g. at least 80% overall sequence identity) to a target sequence or complement thereof that the composition is intended to cluster. The primers may also comprise one or more restriction sites.

Preferably, the kit may comprise a clustering composition as described herein.

The kit may further comprise a recombinase as described herein. The recombinase may be provided separately from the (clustering) composition. For example, the recombinase may be in a different container to the (clustering) composition.

The kit may further comprise a single-stranded nucleotide binding protein as described herein. The single-stranded nucleotide binding protein may be provided separately from the (clustering) composition. For example, the single-stranded nucleotide binding protein may be in a different container to the (clustering) composition.

The kit may further comprise a polymerase as described herein. The polymerase may be provided separately from the (clustering) composition. For example, the polymerase may be in a different container to the (clustering) composition.

The kit may further comprise a plurality and mixture of nucleotide triphosphate (NTPs) as described herein. The nucleotide triphosphate may be provided separately from the (clustering) composition. For example, the nucleotide triphosphate may be in a different container to the (clustering) composition.

The kit may further comprise an ATP-generating substrate as described herein. The ATP-generating substrate may be provided separately from the (clustering) composition. For example, the ATP-generating substrate may be in a different container to the (clustering) composition.

The kit may further comprise an ATP-generating enzyme as described herein. The ATP-generating enzyme may be provided separately from the (clustering) composition. For example, the ATP-generating enzyme may be in a different container to the (clustering) composition.

The kit may comprise at least one selected from the group consisting of: a recombinase, a single-stranded nucleotide binding protein, a polymerase, nucleotide triphosphates (NTPs), an ATP-generating substrate and an ATP-generating enzyme. Preferably, the kit may comprise at least two selected from the group consisting of: a recombinase, a single-stranded nucleotide binding protein, a polymerase, nucleotide triphosphates (NTPs), an ATP-generating substrate and an ATP-generating enzyme. More preferably, the kit may comprise at least three selected from the group consisting of: a recombinase, a single-stranded nucleotide binding protein, a polymerase, nucleotide triphosphates (NTPs), an ATP-generating substrate and an ATP-generating enzyme. Even more preferably, the kit may comprise at least four selected from the group consisting of: a recombinase, a single-stranded nucleotide binding protein, a polymerase, nucleotide triphosphates (NTPs), an ATP-generating substrate and an ATP-generating enzyme. Yet even more preferably, the kit may comprise at least five selected from the group consisting of: a recombinase, a single-stranded nucleotide binding protein, a polymerase, nucleotide triphosphates (NTPs), an ATP-generating substrate and an ATP-generating enzyme. One or more (e.g. each of these components) may be provided separately from the (clustering) composition. For example, one or more (e.g. each of these components) may be in a different container to the (clustering) composition.

Preferably, the kit further comprises at least one selected from the group comprising a recombinase, NTPs and a single stranded nucleotide binding (SSB) protein. More preferably, the composition further comprises at least two selected from the group comprising a recombinase, NTPs and a single stranded nucleotide binding (SSB) protein. One or more (e.g. each of these components) may be provided separately from the (clustering) composition. For example, one or more (e.g. each of these components) may be in a different container to the (clustering) composition.

Preferably, the kit may comprise a recombinase, NTPs and a single stranded nucleotide binding (SSB) protein. One or more (e.g. each of these components) may be provided separately from the (clustering) composition. For example, one or more (e.g. each of these components) may be in a different container to the (clustering) composition.

Preferably, the kit may comprise a recombinase, a single-stranded nucleotide binding protein, a polymerase, nucleotide triphosphates (NTPs), an ATP-generating substrate and an ATP-generating enzyme. One or more (e.g. each of these components) may be provided separately from the (clustering) composition. For example, one or more (e.g. each of these components) may be in a different container to the (clustering) composition.

The kit may further comprise excipients as described herein. The excipient(s) may be provided separately from the (clustering) composition. For example, the excipient(s) may be in a different container to the (clustering) The kit may further comprise one or more agents for use in preparing a template nucleic acid sequence for clustering and sequencing (i.e. library preparation agents). In one embodiment, the kit may further comprise adaptor sequences. The adaptor sequences may be configured such that they can be ligated onto a nucleic acid template to be sequenced. In some preferred embodiments, the kit may comprise a first adaptor sequence that comprises a sequence according to SEQ ID NO. 1 (P5) or a variant or fragment thereof. In other preferred embodiments, the kit may comprise a second adaptor sequence that comprises a sequence according to SEQ ID NO. 2 (P7) or a variant or fragment thereof. In other preferred embodiments, the kit may comprise a third adaptor sequence that comprises a sequence according to SEQ ID NO. 3 (P5′) or a variant or fragment thereof. In other preferred embodiments, the kit may comprise a fourth adaptor sequence that comprises a sequence according to SEQ ID NO. 4 (P7′) or a variant or fragment thereof. More preferably, the kit may comprise at least two of the group selected from the first adaptor sequence, the second adaptor sequence, the third adaptor sequence and the fourth adaptor sequence. Even more preferably, the kit may comprise at least three of the group selected from the first adaptor sequence, the second adaptor sequence, the third adaptor sequence and the fourth adaptor sequence. Yet even more preferably, the kit may comprise the first adaptor sequence, the second adaptor sequence, the third adaptor sequence and the fourth adaptor sequence. The adaptor sequence(s) (e.g. each of the adaptor sequence(s)) may be provided separately from the (clustering) composition. For example, the adaptor sequence(s) (e.g. each of the adaptor sequence(s)) may be in a different container to the (clustering) composition.

The kit may further comprise a metal cofactor composition. The metal cofactor may be configured to activate one or more enzymes in the composition. For example, the metal cofactor may be configured to activate the recombinase and/or the polymerase. Preferably, the metal cofactor composition comprises magnesium ions (e.g. magnesium acetate, magnesium chloride). The metal cofactor composition may be provided separately from the (clustering) composition. For example, the metal cofactor composition may be in a different container to the (clustering) composition.

The kit may further comprise a solid support, preferably a flow cell. Preferably lawn primers (P5 and P7) are immobilised on the flow cell as described in detail above.

In a further aspect, the present disclosure is directed to use of a clustering composition as described herein, or a kit as described herein, to cluster a nucleic acid template, or sequence a nucleic acid template.

In another aspect there is provided a method of amplifying a nucleic acid template comprising reducing or removing inorganic pyrophosphate produced during clustering.

In another aspect, there is provided a method of improving clustering or increasing the clustering kinetics, the method comprising reducing or removing inorganic pyrophosphate produced during the process of clustering. Improving clustering may mean decreasing the time taken to form a cluster, as defined above and/or increasing the density/signal intensity of a cluster and/or increasing the integrity of the cluster/decreasing sequence-specific errors (i.e. faithful amplification of secondary structures within the genome, such as G-quadraplexes and the like). The improvement may be relative to clustering without the levels of pyrophosphate being reduced. An improvement or increase or decrease as used herein may be at least 20%, 30%, 40%, 50%, 60%, 70%, 80% or 90% or more. As shown in FIG. 2 it is possible to achieve the same level of clustering (or clonal amplification of a target sequence) in about 40 minutes (compared to 60 minutes) with the addition of inorganic pyrophosphatase—a decrease of ˜33%.

In another aspect, there is provided a method of resynthesis or improving resynthesis comprising reducing or removing inorganic pyrophosphate produced during clustering, by adding the composition during the resynthesis step. By re-synthesis is meant the step between the first and second sequencing reads where the template is copied using bridged strand resynthesis to produce a second immobilised template that is complementary to the first.

The method may comprise adding the clustering composition as defined herein, to a sample containing a nucleic acid template to be clustered. The compositions may be added to a sample containing a nucleic acid template to be amplified. In particular, by “adding” may mean that the compositions are added to a flow cell before, after or at the same time as a sample containing the nucleic acid template. The nucleic acid template may contain the adaptor sequences (comprising at least one of P5, P5′, P7 and P7′) as described above.

The method may comprise performing nucleic acid clustering at a temperature of about 50° C. to about 75° C., preferably about 55° C. to about 70° C., or more preferably about 60° C. to about 65° C., for example, clustering may be conducted at about 50° C., about 55° C., about 60° C., about 65° C., about 70° C., or about 75° C. This is called thermophilic clustering, and as described above, allows for increased clustering kinetics, decreased clustering times and faster end to end times for the user. Preferably, amplification may be carried out isothermally.

The method may comprise adding the clustering composition only once. That is only one push of the n composition is required to generate a clonal cluster of sufficient density for later sequencing. Alternatively, the composition may be added more than once—i.e. two or more times.

Amplification may be conducted by exclusion amplification. Amplification may be conducted by bridge amplification. In one embodiment, amplification may not be real-time PCR.

In a further embodiment, the present disclosure is directed to a method of sequencing a nucleic acid sequence, wherein the method comprises a step of amplifying a nucleic acid template as described herein; and sequencing the amplified nucleic acid template.

The step of sequencing the amplified nucleic acid template may comprise performing a single read. In other embodiments, the step of sequencing the amplified nucleic acid template comprises performing a paired-end read.

The step of sequencing the amplified nucleic acid template may comprise conducting a first sequencing read and a second sequencing read.

The step of sequencing the amplified nucleic acid template may be conducted using a sequencing-by-synthesis technique or a sequencing-by-ligation technique. Preferably, the step of sequencing the amplified nucleic acid template pay be conducted using a sequencing-by-synthesis technique.

The method of sequencing a nucleic acid sequence may be conducted isothermally.

One or more steps in the method of sequencing a nucleic acid sequence are conducted at a temperature of about 50° C. to about 75° C., about 55° C. to about 70° C., or about 60° C. to about 65° C. for example, one or more steps may be conducted at about 50° C., about 55° C., about 60 ° C., about 65° C., about 70° C., or about 75° C. Preferably, all steps in the method of sequencing a nucleic acid sequence are conducted at a temperature of about 50° C. to about 75° C., about 55° C. to about 70° C., or about 60° C. to about 65° C.; for example, all steps may be conducted at about 50° C., about 55° C., about 60° C., about 65° C., about 70° C., or about 75° C.

Where bridge amplification is used, the step of sequencing the amplified nucleic acid template may comprise a first linearisation step. The first linearisation step may be conducted after (e.g. immediately after) the step of amplifying a nucleic acid template.

The step of sequencing the amplified nucleic acid template may comprise a step of adding an exonuclease. The step of adding an exonuclease may be conducted after the step of amplifying a nucleic acid template. For example, the step of adding an exonuclease may be conducted after (e.g. immediately after) the first linearisation step.

Preferably, the exonuclease is a thermophilic exonuclease. More preferably, the exonuclease is derived from a thermophilic organism, such as Pyrococcus furious.

Preferably, the exonuclease has an optimum working temperature of about 50° C. to about 75° C., about 55° C. to about 70° C., or about 60° C. to about 65° C.

The step of sequencing the amplified nucleic acid template may comprise a first step of dehybridising (or denaturing) a complementary strand bound to the nucleic acid template with a dehybridisation/denaturation agent. The dehybridisation/denaturation agent may be configured to cause the complementary strand to detach from the nucleic acid template and thereby allow the complementary strand to be washed away. The first step of dehybridising a complementary strand may be conducted after the step of amplifying a nucleic acid template. For example, the first step of dehybridising a complementary strand may be conducted after (e.g. immediately after) the step of adding an exonuclease.

The step of sequencing the amplified nucleic acid template may comprise a first step of hybridising a sequencing primer onto the nucleic acid template. The first step of hybridising a sequencing primer may be conducted after the step of amplifying a nucleic acid template. For example, the first step of hybridising a sequencing primer may be conducted after (e.g. immediately after) the first step of dehybridising a complementary strand.

The step of sequencing the amplified nucleic acid template may comprise a first step of performing sequencing-by-synthesis. The first step of performing sequencing-by-synthesis may be conducted after the step of amplifying a nucleic acid template. For example, the first step of performing sequencing-by-synthesis may be conducted after (e.g. immediately after) the first step of hybridising a sequencing primer.

Where a second sequencing read (e.g. for a paired-end read) is conducted, the step of sequencing the amplified nucleic acid may further comprise a step of removing a blocking group from a hydroxyl group of a primer (e.g. a P5 or a P7 lawn primer). For example, the step of removing a blocking group may involve removal of a phosphate group using a blocking group phosphatase. The step of removing a blocking group may be conducted after the step of amplifying a nucleic acid template. For example, the step of removing a blocking group may be conducted after (e.g. immediately after) the first step of performing sequencing-by-synthesis.

Preferably, the blocking group phosphatase is a thermophilic phosphatase. More preferably, the blocking group phosphatase is derived from a thermophilic organism, such as Pyrococcus furious.

Preferably, the phosphatase has an optimum working temperature of about 50° C. to about 75° C., about 55° C. to about 70° C., or about 60° C. to about 65° C.

Where a second sequencing read (e.g. for a paired-end read) is conducted, the step of sequencing the amplified nucleic acid may further comprise a step of generating a complementary version of the amplified nucleic acid template. The step of generating a complementary version of the amplified nucleic acid template may involve using amplification methods as described herein, for example using an ATP-generating substrate and/or an ATP-generating substrate as described herein; preferably creatine kinase and/or creatine phosphate. The step of generating a complementary version of the amplified nucleic acid template may be conducted after the step of amplifying a nucleic acid template. For example, the step of generating a complementary version of the amplified nucleic acid template may be conducted after (e.g. immediately after) the step of removing a blocking group.

Where a second sequencing read (e.g. for a paired-end read) is conducted, the step of sequencing the amplified nucleic acid template may comprise a second linearisation step. The second linearisation step may involve the use of an oxoguanine glycosylase (Ogg). The second linearisation step may be conducted after (e.g. immediately after) the step of generating a complementary version of the amplified nucleic acid template.

Preferably, the oxoguanine glycosylase is a thermophilic oxoguanine glycosylase. More preferably, the oxoguanine glycosylase is derived from a thermophilic organism, such as Methanococcus jannaschii.

Where a second sequencing read (e.g. for a paired-end read) is conducted, the step of sequencing the amplified nucleic acid template may comprise a second step of dehybridising a complementary strand bound to the (complementary version of the) nucleic acid template with a dehybridisation agent. The dehybridisation agent may be configured to cause the complementary strand to detach from the (complementary version of the) nucleic acid template and thereby allow the complementary strand to be washed away. The second step of dehybridising a complementary strand may be conducted after the step of amplifying a nucleic acid template. For example, the second step of dehybridising a complementary strand may be conducted after (e.g. immediately after) the second linearisation step.

Where a second sequencing read (e.g. for a paired-end read) is conducted, the step of sequencing the amplified nucleic acid template may comprise a second step of hybridising a sequencing primer onto the (complementary version of the) nucleic acid template. The second step of hybridising a sequencing primer may be conducted after the step of amplifying a nucleic acid template. For example, the second step of hybridising a sequencing primer may be conducted after (e.g. immediately after) the second step of dehybridising a complementary strand.

Where a second sequencing read (e.g. for a paired-end read) is conducted, the step of sequencing the amplified nucleic acid template may comprise a second step of performing sequencing-by-synthesis. The second step of performing sequencing-by-synthesis may be conducted after the step of amplifying a nucleic acid template. For example, the second step of performing sequencing-by-synthesis may be conducted after (e.g. immediately after) the second step of hybridising a sequencing primer.

The present disclosure will now be described by way of the following non-limiting examples.

### EXAMPLES

### Example 1: FIGS. 2A-2H

Cluster generation was performed utilizing the cBOT or cBOT 2 System with custom recipes (attached). The custom recipes were used in time course studies to examine the reaction kinetics in the presence and absence of Escherichia coli (Eco) inorganic pyrophosphatase (PPiase). The cluster generation workflow was separate seed hybridization followed by amplification driven by the recipe. TruSeq Nano 350 (NA12878; source genomic DNA) supplemented with 1% PhiX v3 Control at a concentration of 300 pM was the seeded library.

Five independent HiSeqX v2.5 flowcells were clustered. Each HiSeqX v2.5 flowcell has eight addressable lanes. The lane layout was as follows: lane 1: control standard ExAmp (2 pushes at 30 minutes each push); lane 2 ExAmp plus 1.2 U Eco PPiase per 100 μl of ExAmp clustering mix (2 pushes at 30 minutes each push); lanes 3-5 triplicate conditions 20-minute control ExAmp (2 pushes at 20 minutes each push); and lanes 6-8 triplicate conditions ExAmp plus 1.2 U Eco PPiase per 100 μl of ExAmp clustering mix (2 pushes at 20 minutes each push). To terminate the clustering reaction at 20 minutes the cBOT manifold lines were physically cut and a syringe was attached to the liberated manifold tubing. 500 μl of HT2 buffer was flushed into the flowcell lane via the syringe. Subsequently, 500 μl of HT1 buffer was flushed into the flowcell lane via the syringe. A fresh manifold was exchanged, and the recipe proceeded to execute the linearization step 1, sequencing primer hybridization, and first base incorporation. A fluorescent scan was taken of the flowcell post first base incorporation in the Cy3 and Cy5 channels set with the PMT at 450 at 50 μM resolution.

Next, a 2×151 sequencing run was executed for each of the five flowcells. Primary metrics were pulled from sequence analysis viewer (SAV). The run was analyzed through the BaseSpace analysis workflow with DRAGEN Germline Alignment v3.7.5, downsample-bam, Firebrand R&D, which was automated with a wrapper in the AVATAR platform. Prism GraphPad v 9.3.1 was utilized for statistical analysis for pairwise comparisons with a confidence interval set at <0.05 for significance.

### Example 2: FIGS. 3A-3H

On board cluster generation (OBCG) was performed utilizing the NextSeq 2000 with a custom recipe to pull the ExAmp supplemented with 0.3 U PPiase per 100 μl clustering reagent or 1.2 U PPiase per 100 μl clustering reagent from a unique position within the sequencing cartridge. TruSeq Nano 450 (NA12878; source genomic DNA) supplemented with 1% PhiX v3 Control at a concentration of 300pM was the seeded library. Two high output (HO) P3 flowcells and accompanying cartridges were utilized for each test condition. A single high output (HO) P3 flowcell was utilized as a control for comparison. A 2×151 sequencing run was executed for each of the flowcells. Primary metrics were pulled from sequence analysis viewer (SAV). The run was analyzed through the BaseSpace analysis workflow with DRAGEN Germline Alignment v3.7.5, downsample-bam, Firebrand R&D, which was automated with a wrapper in the AVATAR platform.

### Example 3: FIGS. 4A-4B

Cluster generation was performed utilizing the cBOT or cBOT 2 System with custom recipes (attached). The custom recipes were used in time course studies to examine the reaction kinetics in the presence and absence of Escherichia coli (Eco) inorganic pyrophosphatase (PPiase). The cluster generation workflow was separate seed hybridization followed by amplification driven by the recipe. TruSeq Nano 350 (NA12878; source genomic DNA) supplemented with 1% PhiX v3 Control at a concentration of 300pM was the seeded library.

A single push 90-minute time course study of the clustering formulation in the presence and absence of PPiase. A.) A fluorescent scan was taken of the flowcell post first base incorporation in the Cy3 and Cy5 channels set with the PMT at 450 at 50 μM resolution of the HiSeqXv2.5 flowcell with lanes 1-8 annotated as follows: 1.) 30 min×2 control 2.) 1×90 min with buffer blank 3.) 1×90 min; 4.) 1×90 min; 5.) 1×90 min 6.) 1×90 min with 0.3 U PPiase per 100 ul of clustering reagent); 7.) 1×90 min with 0.3 U PPiase per 100 ul of clustering reagent); 8.) 1×90 min with 0.3 U PPiase per 100 ul of clustering reagent). To terminate the clustering reaction at the annotated time points the cBOT manifold lines were physically cut and a syringe was attached to the liberated manifold tubing. 500 μl of HT2 buffer was flushed into the flowcell lane via the syringe. Subsequently, 500 μl of HT1 buffer was flushed into the flowcell lane via the syringe. A fresh manifold was exchanged, and the recipe proceeded to execute the linearization step 1, sequencing primer hybridization, and first base incorporation. A single HiSeqX v2.5 flowcells was clustered.

Next, a 2×151 sequencing run was executed for each of the four flowcells. Primary metrics were pulled from sequence analysis viewer (SAV). The run was analyzed through the BaseSpace analysis workflow with DRAGEN Germline Alignment v3.7.5, downsample-bam, Firebrand R&D, which was automated with a wrapper in the AVATAR platform. Prism GraphPad v 9.3.1 was utilized for statistical analysis for pairwise comparisons with a confidence interval set at <0.05 for significance.

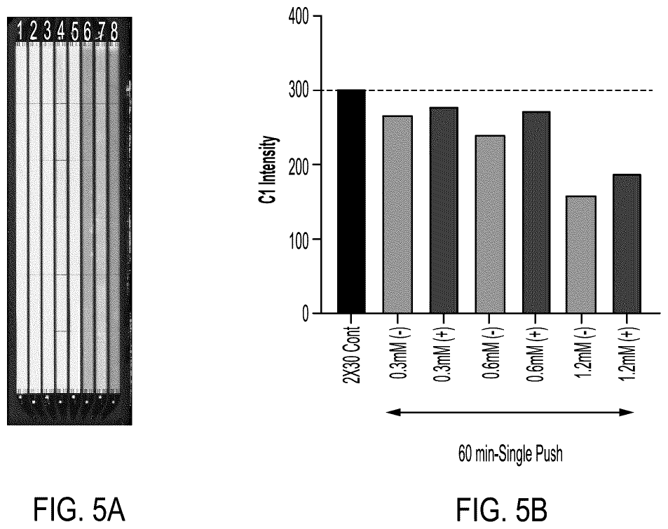

### Example 5: FIGS. 5A-5B

Cluster generation was performed utilizing the cBOT or cBOT 2 System with custom recipes (attached). The custom recipes were used in time course studies to examine the reaction kinetics in the presence and absence of Escherichia coli (Eco) inorganic pyrophosphatase (PPiase). The cluster generation workflow was separate seed hybridization followed by amplification driven by the recipe. TruSeq Nano 350 (NA12878; source genomic DNA) supplemented with 1% PhiX v3 Control at a concentration of 300 pM was the seeded library.

A HiSeqX v2.5 flowcell was clustered. Each HiSeqX v2.5 flowcell has eight addressable lanes. The lane layout was as follows: lane 1: control standard ExAmp (2 pushes at 30 minutes each push); lane 2: ExAmp formulated with 0.3 mM dNTPs; lane 3: ExAmp formulated with 0.3 mM dNTPs & 1.2 U PPiase per 100 μl of ExAmp clustering mix; lane 4: ExAmp formulated with 0.6 mM dNTPs; lane 5: ExAmp formulated with 0.6 mM dNTPs & 1.2 U PPiase per 100 μl of ExAmp clustering mix; lane 6: ExAmp formulated with 1.2 mM dNTPs; lane 7: ExAmp formulated with 1.2 mM dNTPs & 1.2 U PPiase per 100 μl of ExAmp clustering mix; lane 8: ExAmp formulated with 2.4 mM dNTPs & 1.2 U PPiase per 100 μl of ExAmp clustering mix. To terminate the clustering reaction at 60 minutes the cBOT manifold lines were physically cut and a syringe was attached to the liberated manifold tubing. 500 μl of HT2 buffer was flushed into the flowcell lane via the syringe. Subsequently, 500 μl of HT1 buffer was flushed into the flowcell lane via the syringe. A fresh manifold was exchanged, and the recipe proceeded to execute the linearization step 1, sequencing primer hybridization, and first base incorporation. A fluorescent scan was taken of the flowcell post first base incorporation in the Cy3 and Cy5 channels set with the PMT at 450 at 50 μM resolution.

Next, a 2×151 sequencing run was executed for the flowcell. Primary metrics were pulled from sequence analysis viewer (SAV).

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the embodiments described herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

SEQUENCE LISTING
SEQ ID NO: 1:
P5 sequence:
AATGATACGGCGACCACCGAGATCTACAC
SEQ ID NO: 2
P7 sequence:
CAAGCAGAAGACGGCATACGAGAT
SEQ ID NO: 3
P5′ sequence (complementary to P5):
GTGTAGATCTCGGTGGTCGCCGTATCATT
SEQ ID NO: 4
P7′ sequence (complementary to P7):
ATCTCGTATGCCGTCTTCTGCTTG
SEQ ID NO: 5:
MSLLNVPAGKDLPEDIYVVIEIPANADPIKYEIDKESGAL
FVDRFMSTAMFYPCNYGYINHTLSLDGDPVDVLVPTPYPL
QPGSVIRCRPVGVLKMTDEAGEDAKLVAVPHSKLSKEYDH
IKDVNDLPELLKAQIAHFFEHYKDLEKGKWVKVEGWENAE
AAKAEIVASFERAKNK
SEQ ID NO: 6:
MANLKSLPVGDKAPEVVHMVIEVPRGSGNKYEYDPDLGAI
KLDRVLPGAQFYPGDYGFIPSTLAEDGDPLDGLVLSTYPL
LPGVVVEVRVVGLLLMEDEKGGDAKVIGVVAEDQRLDHIQ
DIGDVPEGVKQEIQHFFETYKALEAKKGKWVKVTGWRDRK
AALEEVRACIARYKG
SEQ ID NO: 7:
MNPFHDLEPGPEVPEVVYALIEIPKGSRNKYELDKKSGLI
KLDRVLYSPFYYPVDYGIIPQTWYDDDDPFDIMVIMREPT
YPGVLIEARPIGLFKMIDSGDKDYKVLAVPVEDPYFNDWK
DISDVPKAFLDEIAHFFQRYKELQGKEIIVEGWENAEKAK
QEILRAIELYKEKFKK
SEQ ID NO: 8:
MNPFHDLEPGPDVPEVVYAIIEIPKGSRNKYELDKKTGLL
KLDRVLYSPFFYPVDYGIIPRTWYEDDDPFDIMVIMREPV
YPLTIIEARPIGLFKMIDSGDKDYKVLAVPVEDPYFKDWK
DIDDVPKAFLDEIAHFFKRYKELQGKEIIVEGWEGAEAAK
REILRAIEMYKEKFGKKE
SEQ ID NO: 9:
MMNLWKDLEPGPNPPDVVYAVIEIPRGSRNKYEYDEERGF
FKLDRVLYSPFHYPLDYGFIPRTLYDDGDPLDILVIMQDP
TFPGCVIEARPIGLMKMLDDSDQDDKVLAVPTEDPRFKDV
KDLDDVPKHLLDEIAHMFSEYKRLEGKETEVLGWEGADAA
KEAIVHAIELYEEEHG
SEQ ID NO: 10: RB32 UvsX with His tag:
MGSSHHHHHHSSGLVPRGSHMSIADLKSRLIKASTSKMTA
ELTTSKFFNEKDVIRTKIPMLNIAISGAIDGGMQSGLTIF
AGPSKHFKSNMSLTMVAAYLNKYPDAVCLFYDSEFGITPA
YLRSMGVDPERVIHTPIQSVEQLKIDMVNQLEAIERGEKV
IVFIDSIGNMASKKETEDALNEKSVADMTRAKSLKSLFRI
VTPYFSIKNIPCVAVNHTIETIEMFSKTVMTGGTGVMYSA
DTVFIIGKRQIKDGSDLQGYQFVLNVEKSRTVKEKSKFFI
DVKFDGGIDPYSGLLDMALELGFVVKPKNGWYAREFLDEE
TGEMIREEKSWRAKDTNCTTFWGPLFKHQPFRDAIKRAYQ
LGAIDSNEIVEAEVDELINSKVEKFKSPESKSKSAADLET
DLEQLSDMEEFNEGGHHHHH
SEQ ID NO: 11 RB32 UvsX:
MSIADLKSRLIKASTSKMTAELTTSKFFNEKDVIRTKIPM
LNIAISGAIDGGMQSGLTIFAGPSKHFKSNMSLTMVAAYL
NKYPDAVCLFYDSEFGITPAYLRSMGVDPERVIHTPIQSV
EQLKIDMVNQLEAIERGEKVIVFIDSIGNMASKKETEDAL
NEKSVADMTRAKSLKSLFRIVTPYFSIKNIPCVAVNHTIE
TIEMFSKTVMTGGTGVMYSADTVFIIGKRQIKDGSDLQGY
QFVLNVEKSRTVKEKSKFFIDVKFDGGIDPYSGLLDMALE
LGFVVKPKNGWYAREFLDEETGEMIREEKSWRAKDTNCTT
FWGPLFKHQPFRDAIKRAYQLGAIDSNEIVEAEVDELINS
KVEKFKSPESKSKSAADLETDLEQLSDMEEFNE
SEQ ID NO: 12 Thermophilic UvsX HQ:
MSIADLKSRLIKASTSKMTAELTTSKFFNEKDVIRTKIPM
LNIAISGAIDGGMQSGLTIFAGPSKSFKSNMSLTMVAAYL
NKYPDAVCLFYDSEFGITPAYLRSMGVDPERVIHTPIQSV
EQLKIDMVNQLEAIERGEKVIVFIDSIGNMASKKETEDAL
NEKSVADMTRAKSLKSLFRIVTPYFSIKNIPCVAVNHTIE
TIEMFSKTVMTGGTGVMYSADTVFIIGKRQIKDGSDLQGY
QFVLNVEKSRTVKEKSKFFIDVKFDGGIDPYSGLLDMALE
LGFVVKPKNGWYAREFLDEETGEMIREEKSWRAKDINCTT
FWGPLFKHQPFRDAIKRAYQLGAIDSNEIVEAEVDELINS
KVEKFKSPESKSKSAADLETDLEQLSDMEEFNEHQHQH
SEQ ID NO: 13 Thermophilic UvsX His:
MSIADLKSRLIKASTSKMTAELTTSKFFNEKDVIRTKIPM
LNIAISGAIDGGMQSGLTIFAGPSKSFKSNMSLTMVAAYL
NKYPDAVCLFYDSEFGITPAYLRSMGVDPERVIHTPIQSV
EQLKIDMVNQLEAIERGEKVIVFIDSIGNMASKKETEDAL
NEKSVADMTRAKSLKSLFRIVTPYFSIKNIPCVAVNHTIE
TIEMFSKTVMTGGTGVMYSADIVFIIGKRQIKDGSDLQGY
QFVLNVEKSRTVKEKSKFFIDVKFDGGIDPYSGLLDMALE
LGFVVKPKNGWYAREFLDEETGEMIREEKSWRAKDINCTT
FWGPLFKHQPFRDAIKRAYQLGAIDSNEIVEAEVDELINS
KVEKFKSPESKSKSAADLETDLEQLSDMEEFNEGGHHHHH

1. A clustering composition comprising an inorganic pyrophosphatase.

2. The composition of claim 1, wherein the composition comprises inorganic pyrophosphatase at a concentration of about 0.01 μM to about 1000 μM.

3. The composition of claim 1, wherein the composition further comprises at least one selected from the group consisting of: a recombinase, a single-stranded nucleotide binding protein, a polymerase, nucleotide triphosphates (NTPs), an ATP-generating substrate and an ATP-generating enzyme.

4. (canceled)

5. The composition of claim 3, wherein the polymerase is DNA Polymerase I and the recombinase is Recombinase A.

6. The composition of claim 1, wherein the composition does not comprise PEG.

7. The composition of claim 1, wherein the composition comprises a buffer, and wherein the composition is buffered to a pH of about 6.0 to about 9.0.

8. The composition of claim 1, wherein the composition is a resynthesis composition.

9. A thermophilic clustering composition, wherein the composition comprises a thermophilic inorganic pyrophosphatase.

10. A mesophilic clustering composition wherein the composition comprises a mesophilic inorganic pyrophosphatase.

11. A kit comprising the clustering composition of claim 1.

12. The kit of claim 11, wherein the kit further comprises a metal cofactor composition, wherein the metal cofactor composition comprises magnesium ions.

13. The clustering composition of claim 1, wherein the composition does not comprise primers having a length of between 18 to 22 base pairs.

14. Use of the clustering composition of claim 1 to amplify a nucleic acid sequence.

15. A method of amplifying a target nucleic acid template, the method comprising reducing or removing inorganic pyrophosphate during clustering.

16. (canceled)

17. The method of claim 15, wherein the method comprises adding the clustering composition according to claim 1.

18. The method of claim 17, wherein nucleic acid clustering is performed at a temperature of about 50° C. to about 75° C.

19. (canceled)

20. A method of sequencing a nucleic acid sequence, wherein the method comprises:
amplifying a nucleic acid template using the method of claim 15; and
sequencing the amplified nucleic acid template.

21. The method according to claim 20, wherein the step of sequencing the amplified nucleic acid template comprises conducting a first sequencing read and a second sequencing read.

22. The method according to claim 20, wherein the step of sequencing the amplified nucleic acid template is conducted using a sequencing-by-synthesis technique or a sequencing-by-ligation technique.

23. The method of claim 20, wherein the method is conducted at temperatures of about 50° C. to about 75° C.
